(19) United States
(12) Patent Application Publication
(10) Pub. No.: US 2008/0058515 A1
(43) Pub. Date: Mar. 6, 2008

(54) Chemical Compounds

(76) Inventors: David Harold Drewry, Durham, NC (US); James Andrew Linn, Cary, NC (US); James Marvin Veal, Apex, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/566,040

(22) PCT Filed: Jul. 23, 2004
(86) PCT No.: PCT/US04/23680

§ 371 Date: Jan. 26, 2006

Related U.S. Application Data
(60) Provisional application No. 60/490,828, filed on Jul. 29, 2003.

(51) Int. Cl. C07D 211/32; C07D 215/00; C07D 231/56; C07D 261/04; C07D 401/00; C07D 403/00; C07D 413/00; C07D 417/00; C07D 471/10

(52) U.S. Cl. 544/116; 544/359; 544/371; 546/152; 546/199; 546/20; 546/275.7; 548/159; 548/245; 548/362.5

(57) ABSTRACT

Indazolylacrylamide derivatives, which are useful as SGK-1 inhibitors are described herein. The described invention also includes methods of making such indazolylacrylamide derivatives as well as methods of using the same in the treatment of diseases mediated by inappropriate SGK-1 activity.

### BACKGROUND OF THE INVENTION

The present invention relates to indazolylacrylamide derivatives, compositions and medicaments containing the same, as well as processes for the preparation and use of such compounds, compositions and medicaments. Such indazolylacrylamide derivatives are useful in the treatment of diseases associated with inappropriate SGK-1 activity.

An important large family of enzymes is the protein kinase enzyme family. Currently, there are about 400 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

Serum and Glucocorticoid-Regulated Kinase 1 (SGK-1) is a serine/threonine protein kinase, whose function is thought linked to cell proliferation and electrolyte homeostasis. SGK-1 is a member of a family of intracellular kinases which includes protein kinase B. While it is transcriptionally induced by glucocorticoids and mineralocorticoids, it is activated by insulin and IGF-1 mediated phosphorylation through PI3-kinase and PDK-1. SGK-1 is thought to mediate several mechanisms, which contribute to disease states. As indicated above, IGF-1 activates SGK-1 and is involved in fibronectin synthesis, an element of renal fibrosis. Consequently, SGK-1 may mediate IGF-1 action on fibronectin synthesis. The anti-diuretic aldosterone induces expression of SGK-1, which in turn activates the epithelial Na⁺ channel thereby affecting Na⁺ transport. Accordingly, SGK-1 may serve to mediate aidosterone-induced Na⁺ retention in renal and cardiovascular disease. SGK-1 may also mediate repair processes involving cell proliferation, for instance, through thrombin. Thrombin causes renal cell proliferation and increases SGK-1 expression in renal cells. Therefore, SGK-1 may provide a novel therapy for the regulation of electrolyte balance in renal and cardiovascular disease and in damaging cell proliferation in renal disease.

The present inventors have discovered novel indazolylacrylamide compounds, which are inhibitors of SGK-1 activity. Such indazolylacrylamide derivatives are useful in the treatment of disorders associated with inappropriate SGK-1 activity.

### BRIEF SUMMARY OF THE INVENTION

In one aspect of the present invention, there is provided a compound of Formula (I):

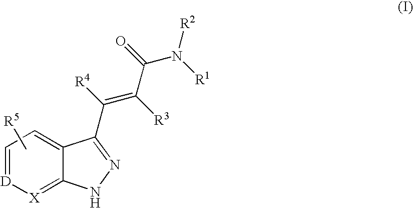

or a salt, solvate, or physiologically functional derivative thereof:
wherein:

### D is C—R and X is N, or

### D is N and X is C—R, or

### D is C—R and X is C—R,

where each R is independently selected from hydrogen, halo, cyano, or C₁-C₆ alkyl;

### R¹ is a group defined by -(Q)ₘ-(Q¹)ₙ-(Q²)ₚ, wherein;

Q is arylene or heteroarylene, and m is 0 or 1,
Q¹ is O(CH₂)_{q}, (CH₂)ᵣC(O), or S(O)₂, and
   n is 0 or 1,
   q is 0, 1, 2, 3, or 4, and
   r is 1, 2, 3, or 4,
Q² is C₁-C₆ alkyl, C₃-C₇ cycloalkyl, —OH, C₁-C₃ alkoxy, NR⁶R⁷, aryl, aryloxy, heteroaryl, heterocyclyl, or R⁹R¹⁰, and p is 0 or 1

### R² is —H, or C₁-C₆ alkyl; or

### R¹ and R² together with the nitrogen to which they are attached form a ring system, said ring system being a substituted or unsubstituted heterocyclyl or heterocyclic spiro ring system;

### R³ is —H or C₁-C₃ alkyl;

### R⁴ is —H or C₁-C₃ alkyl;

### R⁵ is —H, halo, —CN, —OH, C₁-C₃ alkyl, C₁-C₃alkoxy, —NO₂, aryl, or NR′R″;

### R⁶ is —H or C₁-C₆ alkyl;

### R⁷ is —H, C₁-C₆ alkyl, C₃-C₇ cycloalkyl, —R⁹R¹⁰, aralkyl, heterocyclyl, or —C(O)R⁸;

### R⁸ is hydrogen or C₁-C₆ alkyl;

### R⁹ is C₁-C₆ alkylene or heterocyclylene;

### R¹⁰ is C₁-C₆ alkoxy, aryl, aralkyl, heteroaryl, aryloxy, heterocyclyl, —C(O)OR⁸, —C(O)R⁸, or —C(O)NR′R″;

### R′ is —H or C₁-C₆ alkyl;

### R″ is —H, C₁-C₆ alkyl, —C(O)R′″, —S(O)₂R′″, or C(O)NHR′″; and

### R′″ is C₁-C₆ alkyl, aryl, aralkyl, heteroaryl, or heterocyclyl.

In a second aspect of the present invention, there is provided a pharmaceutical composition comprising a therapeutically effective amount of a compound of formula (I) or a salt, solvate, or a physiologically functional derivative thereof and one or more of pharmaceutically acceptable carriers, diluents and excipients.

In a third aspect of the present invention, there is provided a method of treating a disorder in a mammal, said disorder being mediated by inappropriate SGK-1 activity, comprising: administering to said mammal a therapeutically effective amount of a compound of formula (I) or a salt, solvate or a physiologically functional derivative thereof.

In a fourth aspect of the present invention, there is provided a compound of formula (I), or a salt, solvate, or a physiologically functional derivative thereof for use in therapy.

In an fifth aspect of the present invention, there is provided the use of a compound of formula (I), or a salt, solvate, or a physiologically functional derivative thereof in the preparation of a medicament for use in the treatment of a disorder mediated by inappropriate SGK-1 activity.

### DETAILED DESCRIPTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having from one to twelve carbon atoms, optionally substituted with substituents selected from the group consisting of unsubstituted C₁-C₆ alkyl, C₁-C₆ hydroxyalkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aryl, aryloxy, heteroaryl, aminosulfonyl optionally substituted by alkyl, nitro, cyano, halo, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₃ alkyl” and “C₁-C₆ alkyl” refer to an alkyl group, as defined above, containing at least 1, and at most 3 or 6 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, and n-hexyl.

As used herein, the term “alkylene” refers to a straight or branched chain divalent hydrocarbon radical having from one to ten carbon atoms, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halo, and C₁-C₆ perfluoroalkyl, multiple degrees of substitution being allowed. Examples of “alkylene” as used herein include, but are not limited to, methylene, ethylene, n-propylene, n-butylene, and the like.

As used herein, the terms “C₁-C₃ alkylene” and “C₁-C₆ alkylene” refer to an alkylene group, as defined above, which contains at least 1, and at most 3 or 6, carbon atoms respectively. Examples of “C₁-C₆ alkylene” and “C₁-C₆ alkylene” groups useful in the present invention include, but are not limited to, methylene, ethylene, n-propylene, n-butylene, isopentylene, and the like.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “C₁-C₆ haloalkyl” refers to an alkyl group as defined above containing at least 1, and at most 6 carbon atoms respectively substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “cycloalkyl” refers to a non-aromatic cyclic hydrocarbon ring containing from 3 to 10 carbon atoms and which optionally includes a C₁-C₆ alkyl linker through which it may be attached. In a like manner the term “C₃-C₇ cycloalkyl” refers to a non-aromatic cyclic hydrocarbon ring having from three to seven carbon atoms and which optionally includes a C₁-C₆ alkyl linker through which it may be attached. The C₁-C₆ alkyl group is as defined above. Exemplary “C₃-C₇ cycloalkyl” groups useful in the present invention include, but are not limited to, cyclopropyl, cyclobutyl, cyclopentyl, cyclohexyl and cycloheptyl.

As used herein, the term “heterocyclic” or the term “heterocyclyl” refers to a three to twelve-membered non-aromatic heterocyclic ring, being saturated or having one or more degrees of unsaturation, containing one or more heteroatom substitutions selected from S, S(O), S(O)₂, O, or N, optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halo, aryl, aralkyl, heteroaryl, or C₁-C₆ perfluoroalkyl, multiple degrees of substitution being allowed. Such a ring may be optionally fused to one or more other “heterocyclic” ring(s) or cycloalkyl ring(s). Examples of “heterocyclic” moieties include, but are not limited to, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

As used herein the term “heterocyclic spiro ring system” or “heterocyclyl spiro ring system” refers to a ring system having a three to twelve-membered non-aromatic heterocyclic ring, being saturated or having one or more degrees of unsaturation, containing one or more heteroatom substitutions selected from S, S(O), S(O)₂, O, or N, and a further ring being a heterocyclic, or aryl, or heteroaryl, or cycloalkyl ring, said rings of said ring system having one atom in common and being optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halo, aryl, aralkyl, heteroaryl, or C₁-C₆ perfluoroalkyl, multiple degrees of substitution being allowed. Examples of “heterocyclic spiro ring systems” moieties include, but are not limited to, triazaspiro[4.5]decan-4-one.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include C₁-C₆ alkyl, C₁-C₆ alkoxy, C₁-C₆ haloalkyl, C₁-C₆ haloalkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino optionally substituted by alkyl or acyl, carboxy, tetrazolyl, carbamoyl optionally substituted by alkyl, aryl, or heteroaryl, aminosulfonyl optionally substituted by alkyl, acyl, aroyl, aroylamino, heteroaroyl, acyloxy, aroyloxy, heteroaroyloxy, alkoxycarbonyl, nitro, cyano, halo, heteroaryl, heterocyclyl, aryl optionally substituted with aryl, halo, C₁-C₆ alkyl, C₁-C₆ haloalkyl, or C₁-C₆ alkylsulfonyl, ureido, arylurea, alkylurea, cycloalkylurea, alkylthiourea, aryloxy, or aralkoxy, multiple degrees of substitution being allowed. Examples of “aryl” groups include, but are not limited to, phenyl, 2-naphthyl, 1-naphthyl, biphenyl, as well as substituted derivatives thereof.

As used herein, the term “arylene” refers to a benzene ring diradical or to a benzene ring system diradical fused to one or more optionally substituted benzene rings, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halo, and C₁-C₆ perfluoroalkyl, multiple degrees of substitution being allowed. Examples of “arylene” include, but are not limited to, benzene-1,4-diyl, naphthalene-1,8-diyl, anthracene-1,4-diyl, and the like.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl-3-isoxazolylmethyl, and 2-imidazolyl ethyl.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halo, C₁-C₆ perfluoroalkyl, heteroaryl, or aryl, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, and substituted versions thereof.

As used herein, the term “heteroarylene” refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents selected from the group consisting of: C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halo, or C₁-C₆ perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “alkoxy” refers to the group RₐO—, where Rₐ is alkyl as defined above and the terms “C₁-C₃ alkoxy” and “C₁-C₆ alkoxy” refer to an alkoxy group as defined herein wherein the alkyl moiety contains at least 1, and at most 3 or 6, carbon atoms. Exemplary “C₁-C₃ alkoxy” and “C₁-C₆ alkoxy” groups useful in the present invention include, but are not limited to, methoxy, ethoxy, n-propoxy, isopropoxy, n-butoxy, and t-butoxy.

As used herein, the term “amino” refers to the group —NH₂.

As used herein the term “alkylamino” refers to the group —NHRₐ wherein Rₐ is alkyl as defined above.

As used herein the term “arylamino” refers to the group —NHRₐ wherein Rₐ is aryl as defined above.

As used herein the term “aralkylamino” refers to the group —NHRₐ wherein Rₐ is an aralkyl group as defined above.

As used herein the term “aralkoxy” refers to the group R_{b}RₐO—, where Rₐ is alkylene and R_{b} is aryl or heteroaryl all as defined above.

As used herein the term “aryloxy” refers to the group RₐO—, where Rₐ is aryl or heteroaryl both as defined above.

As used herein the term “ureido” refers to the group —NHC(O)NH₂

As used herein, the term “arylurea” refers to the group —NHC(O)NHRₐ wherein Rₐ is aryl as defined above.

As used herein, the term, “arylthiourea” refers to the group —NHC(S)NHRₐ wherein Rₐ is aryl as defined above.

As used herein, the term “alkylurea” refers to the group —NHC(O)NHRₐ wherein Rₐ is alkyl as defined above.

As used herein, the term “cycloalkylurea” refers to the group —NHC(O)NHRₐ wherein Rₐ is cycloalkyl as defined above.

As used herein, the term “C₃-C₇ cycloalkoxy” refers to the group RₐO—, where Rₐ is C₃-C₇ cycloalkyl as defined above. Exemplary C₃-C₇ cycloalkoxy groups useful in the present invention include, but are not limited to, cyclobutoxy, and cyclopentoxy.

As used herein, the term “haloalkoxy” refers to the group RₐO—, where Rₐ is haloalkyl as defined above and the term “C₁-C₆ haloalkoxy” refers to a haloalkoxy group as defined herein wherein the haloalkyl moiety contains at least 1, and at most 6, carbon atoms. Exemplary C₁-C₆ haloalkoxy groups useful in the present invention include, but is not limited to, trifluoromethoxy.

As used herein, the term “alkylsulfanyl” refers to the group RₐS—, where Rₐ is alkyl as defined above and the term “C₁-C₆ alkylsulfanyl” refers to an alkylsulfanyl group as defined herein wherein the alkyl moiety contains at least 1, and at most 6, carbon atoms.

As used herein, the term “haloalkylsulfanyl” refers to the group RₐS—, where Rₐ is haloalkyl as defined above and the term “C₁-C₆ haloalkylsulfanyl” refers to a haloalkylsulfanyl group as defined herein wherein the alkyl moiety contains at least 1, and at most 6, carbon atoms.

As used herein, the term “alkylsulfenyl” refers to the group RₐS(O)—, where Rₐ is alkyl as defined above and the term “C₁-C₆ alkylsulfenyl” refers to an alkylsulfenyl group as defined herein wherein the alkyl moiety contains at least 1, and at most 6, carbon atoms.

As used herein, the term “alkylsulfonyl” refers to the group RₐS(O)₂—, where Rₐ is alkyl as defined above and the term “C₁-C₆ alkylsulfonyl” refers to an alkylsulfonyl group as defined herein wherein the alkyl moiety contains at least 1, and at most 6, carbon atoms.

As used herein, the term “alkylsulfonylamino” refers to the group —NHS(O)₂Rₐ wherein Rₐ is alkyl as defined above and the term “C₁-C₆ alkylsulfonylamino” refers to an alkylsulfonylamino group as defined herein wherein the alkyl moiety contains at least 1, and at most 6, carbon atoms.

As used herein, the term “arylsulfonylamino” refers to the group —NHS(O)₂Rₐ wherein Rₐ is aryl as defined above.

As used herein, the term “alkylcarboxyamide” refers to the group —NHC(O)Rₐ wherein Rₐ is alkyl, amino, or amino substituted with alkyl, aryl or heteroaryl as described above.

As used herein the term “alkylcarboxy” refers to the group —C(O)Rₐ wherein Rₐ is alkyl as described above.

As used herein, the term “oxo” refers to the group ═O.

As used herein, the term “mercapto” refers to the group —SH.

As used herein, the term “carboxy” refers to the group —C(O)ORₐ, wherein Rₐ is H or alkyl as defined herein.

As used herein, the term “cyano” refers to the group —CN.

As used herein the term “cyanoalkyl” refers to the group —RₐCN wherein Rₐ is alkyl as defined above. Exemplary “cyanoalkyl” groups useful in the present invention include, but are not limited to, cyanomethyl, cyanoethyl, and cyanoisopropyl.

As used herein, the term “aminosulfonyl” refers to the group —S(O)₂NH₂.

As used herein, the term “carbamoyl” refers to the group —OC(O)NHRₐ. where Rₐ is hydrogen or alkyl as defined herein.

As used herein, the term “carboxamide” refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂—.

As used herein, the term “acyl” refers to the group RₐC(O)—, where Rₐ is alkyl, cycloalkyl, or heterocyclyl as defined herein.

As used herein, the term “aroyl” refers to the group RₐC(O)—, where Rₐ is aryl as defined herein.

As used herein, the term “aroylamino” refers to the group RₐC(O)NH—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

It is understood that due to the presence of the double bond containing alkenylene chain in formula (I), also included within the scope of formula (I) are the respective pure E and Z geometric isomers as well as mixtures of E and Z isomers of the compounds of formula (I). The invention as described and claimed does not set any limiting ratios on prevalence of Z to E isomers.

In one embodiment, the compounds of formula (I) are in the form of a substantially pure E geometric isomer. In another embodiment, the compounds of formula (I) are in the form of a substantially pure Z geometric isomer. In a further embodiment, the compounds of formula (I) are in the form of a mixture of E geometric isomer and Z geometric isomer in any proportions of said geometric isomers.

It is to be understood that reference to compounds of formula (I) above, following herein, refers to compounds within the scope of formula (I) as defined above with respect to D, X, Q, Q¹, Q², R, R¹, R², R³, R⁴, R⁵, R⁶, R⁷, R⁸, R⁹, R¹⁰, R′, R″, and R′″ unless specifically limited otherwise.

In one embodiment, D is N. In a preferred embodiment, D is C—R, wherein R is H.

In one embodiment, X is N. In a preferred embodiment, X is C—R, wherein R is H.

In one embodiment, D is N and X is C—R, wherein R is H. In another embodiment, D is C—R, wherein R is H and X is N. In a preferred embodiment, X is C—R, and D is C—R, wherein each R is H.

As indicated above, R¹ is a group defined by -(Q)ₘ-(Q¹)ₙ-(Q²)ₚ. In one embodiment, m and n are 0, p is 1 and R¹ is -(Q²). In another embodiment, m and p are 1, n is 0 and R¹ is -(Q)-(Q²). In another embodiment, m, n, and p are 1, and R¹ is -(Q)-(Q¹)-(Q²).

In one embodiment, m and p are 1 and Q is arylene. In another embodiment, m and p are 1 and Q is heteroarylene.

In one embodiment, n and p are 1 and Q¹ is O(CH₂)_{q}, where q is 0, 1, 2, 3, or 4, preferably q is 1 or 2. In another embodiment, n and p are 1 and Q¹ is (CH₂)ᵣ and where r is 1, 2, 3, or 4, preferably r is 1 or 2. In still another embodiment, n and p are 1 and Q¹ is C(O). In a further embodiment, n and p are 1 and Q¹ is S(O)₂.

In one embodiment, p is 1 and Q² is NR⁶R⁷, where R¹ and R⁷ are as defined above. In another embodiment, p is 1 and Q² is heteroaryl. In still another embodiment, p is 1 and Q² is aryl. In a further embodiment, p is 1 and Q² is R⁹R¹⁰, where R⁹ and R¹⁰ are as defined above.

In one embodiment, R² is C₁-C₆ alkyl, preferably methyl. In a preferred embodiment R² is —H.

In one embodiment, R³ is C₁-C₆ alkyl, preferably methyl. In a preferred embodiment R³ is —H.

In one embodiment, R⁴ is C₁-C₆ alkyl, preferably methyl. In a preferred embodiment R⁴ is —H.

In one embodiment, R⁵ is —H, C₁-C₃ alkyl, C₁-C₃ alkoxy, or halo. In a preferred embodiment R⁵ is —H.

In one embodiment, R⁶ is —H or C₁-C₆ alkyl. In a preferred embodiment R⁶ is —H.

In one embodiment, R⁷ is C₁-C₆ alkyl, preferably methyl, ethyl or isopropyl. In another embodiment, R⁷ is aralkyl, preferably benzyl. In another embodiment, R⁷ is R⁹R¹⁰, where R⁹ and R¹⁰ are as defined above.

Specific examples of compounds of the present invention include the following:
(2E)-N-(1,3-benzothiazol-6-yl)-3-(1 h-indazol-3-yl)-2-propenamide;
(2E)-N-(3,4-dimethyl-5-isoxazolyl)-3-(1H-indazol-3-yl)-2-propenamide;
(2E)-N-(2-cyanophenyl)-3-(1 h-indazol-3-yl)-2-propenamide;
(2E)-3-(1H-indazol-3-yl)-N-(6-methoxy-3-pyridinyl)-2-propenamide;
(2E)-N-(3-chlorophenyl)-3-(1H-indazol-3-yl)-2-propenamide;
(2E)-N-(2,3-dihydro-1H-inden-5-yl)-3-(1H-indazol-3-yl)-2-propenamide;
(2E)-N—[4-(dimethylamino)phenyl]-3-(1H-indazol-3-yl)-2-propenamide;
(2E)-N-(3-cyclopropyl-1-methyl-1H-pyrazol-5-yl)-3-(1H-indazol-3-yl)-2-propenamide;
(2E)-N—3-(1H-indazol-3-yl)-N-(5-quinolinyl)-2-propenamide;
(2E)-N-[3-(acetylamino)phenyl]-3-(1H-indazol-3-yl)-2-propenamide;
(2E)-3-(1H-indazol-3-yl)-N-(3,4,5-trimethoxyphenyl)-2-propenamide;
(2E)-N-(3-benzoylphenyl)-3-(1H-indazol-3-yl)-2-propenamide;
(2E)-N-[3-chloro-4-(4-morpholinyl)phenyl]-3-(1H-indazol-3-yl)-2-propenamide;
(2E)-N-{5-[(diethylamino)sulfonyl]-2-methoxyphenyl}-3-(1H-indazol-3-yl)-2-propenamide;
(2E)-N-{4-[2-(diisopropylamino)ethoxy]-3-methoxyphenyl}-3-(1-H-indazol-3-yl)-2-propenamide;
(2E)-3-(1H-indazol-3-yl)-N-(3-methoxy-4-{2-[(2-phenoxyethyl)amino]ethoxy}phenyl)-2-propenamide;
(2E)-3-(1H-indazol-3-yl)-N-{3-methoxy-4-[2-(4-morpholinyl)ethoxy]phenyl}-2-propenamide;
(2)-3-(1H-indazol-3-yl)-N-(3-methoxy-4-{2-[(2-methoxyethyl)amino]-ethoxy}phenyl)-2-propenamide;
(2E)-3-(1H-indazol-3-yl)-N-(3-methoxy-4-{2-[methyl(propyl)amino]ethoxy}phenyl)-2-propenamide;
(2E)-N-[4-(2-{[2-(4-chlorophenyl)ethyl]amino}ethyoxy)-3-methoxyphenyl]-3-(1H-indazol-3-yl)-2-propenamide;
Ethyl 4-{[2-(4-{[(2E)-3-(1H-indazol-3-yl)-2-propenoyl]amino}-2-methoxyphenoxy)ethyl]amino}-1-piperidine carboxylate;
(2E)-N-{4-[2-(4-acetyl-1-piperazinyl)ethoxy]-3-methoxyphenyl}-3-(1H-indazol-3-yl)-2-propenamide;
(2E)-N-benzyl-3-(1H-indazol-3-yl)prop-2-enamide;
(2E)-3-(1H-indazol-3-yl)-N-isobutylprop-2-enamide;
(2E)-3-(1H-indazol-3-yl)-N-(3-morpholin-4-ylpropyl)prop-2-enamide;
(2E)-N-[2-(4-chlorophenyl)ethyl]-3-(1H-indazol-3-yl)prop-2-enamide;
ethyl 1-[(2E)-3-(1H-indazol-3-yl)prop-2-enoyl]piperidine-4-carboxylate;
3-[(1E)-3-(4-benzylpiperidin-1-yl)-3-oxoprop-1-enyl]-1H-indazole;
(2E)-N-ethyl-3-(1H-indazol-3-yl)-N-(pyridin-4-ylmethyl)prop-2-enamide;
8-[(2E)-3-(1H-indazol-3-yl)prop-2-enoyl]-1-phenyl-1,3,8-triazaspiro[4.5]decan-4-one;
3-[(1E)-3-oxo-3-(4-pyrazin-2-ylpiperazin-1-yl)prop-1-enyl]-1H-indazole;
Methyl 4-(((2E)-3-(1H-indazol-3-yl)prop-2-enoyl)amino)benzoate;
4-(((2E)-3-(1H-indazol-3-yl)prop-2-enoyl)amino)benzoic acid;
Methyl 3-(((2E)-3-(1H-indazol-3-yl)prop-2-enoyl)amino)benzoate;
3-(((2E)-3-(1H-indazol-3-yl)prop-2-enoyl)amino)benzoic acid;
4-(((2E)-3-(1H-indazol-3-yl)prop-2-enoyl)amino)-N-(2-pyridin-3-ylethyl)benzamide;
4-(((2E)-3-(1H-indazol-3-yl)prop-2-enoyl)amino)-N-methyl-N-(2-pyridin-2-ylethyl)benzamide;
4-(((2E)-3-(1H-indazol-3-yl)prop-2-enoyl)amino)-N-methyl-N-(1-methylpyrrolidin-3-yl)benzamide;
3-(((2E)-3-(1H-indazol-3-yl)prop-2-enoyl)amino)-N-(2-morpholin-4-ylethyl)benzamide;
(2E)-N-(3-((4-benzylpiperazin-1-yl)carbonyl)phenyl)-3-(1H-indazol-3-yl)prop-2-enamide; and
N-ethyl-3-(((2E)-3-(1H-indazol-3-yl)prop-2-enoyl)amino)-N-(pyridin-4-ylmethyl)benzamide
   or a salt, solvate, or physiologically functional derivative thereof.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of formula (I) and salts, solvates and physiological functional derivatives thereof, are believed to have utility in chronic renal disease, congestive heart failure, and cardiovascular remodeling as a result of inhibition of the protein kinase SGK-1.

The present invention thus also provides compounds of formula (I) and pharmaceutically acceptable salts or solvates thereof, or physiologically functional derivatives thereof, for use in medical therapy, and particularly in the treatment of disorders mediated by SGK-1 activity.

The inappropriate SGK-1 activity referred to herein is any SGK-1 activity that deviates from the normal SGK-1 activity expected in a particular mammalian subject. Inappropriate SGK-1 activity may take the form of, for instance, an abnormal increase in activity, or an aberration in the timing and or control of SGK-1 activity. Such inappropriate activity may result then, for example, from overexpression or mutation of the protein kinase leading to inappropriate or uncontrolled activation.

The present invention is directed to methods of regulating, modulating, or inhibiting SGK-1 for the prevention and/or treatment of disorders related to unregulated SGK-1 activity. In particular, the compounds of the present invention can also be used in the treatment of certain forms of renal and cardiovascular disease as well as congestive heart failure.

A further aspect of the invention provides a method of treatment of a mammal suffering from a disorder mediated by SGK-1 activity, which includes administering to said subject an effective amount of a compound of formula (I) or a pharmaceutically acceptable salt, solvate, or a physiologically functional derivative thereof. In a preferred embodiment, the disorder is a susceptible cancer.

A further aspect of the present invention provides the use of a compound of formula (I), or a pharmaceutically acceptable salt or solvate thereof, or a physiologically functional derivative thereof, in the preparation of a medicament for the treatment of a disorder characterized by SGK-1 activity.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Compounds of Formula I can be prepared according to the synthetic sequences illustrated in Schemes 1, 2, and 3 and further detailed in the Examples section following.

Scheme 1 illustrates a synthetic scheme for the preparation of the indazolylacrylamide derivatives of Formula I. In this scheme R¹, R², R³, R⁴, and R⁵ are as defined above. Such methods are described for instance, in Collot et al., Tetrahedron 55, 6917-6922 (1999) and Grehn & Ragnarsson, Angew. Chem. Int. Ed. 23(4), 296-297 (1984).

Scheme 2 illustrates a synthetic scheme for the preparation of 4-(2-bromoethoxy)-3-methoxyaniline (see Intermediate Example 5) which may be utilized as an intermediate (see Scheme 3) in the preparation of indazolylacrylamide derivatives of Formula I. A method for the synthesis of the precursor compound I-(2-bromoethoxy)-2-methoxy-4-nitrobenzene is described in Marquet, J.; Cayon, E.; Martin, X.; Casado, F.; Gallardo, I. J. Org. Chem. 60, 12, 1995, 3814-3825.

Scheme 3 illustrates a synthetic scheme for the preparation of indazolylacrylamide derivatives of Formula I wherein, R⁶ and R⁷ are as defined above.

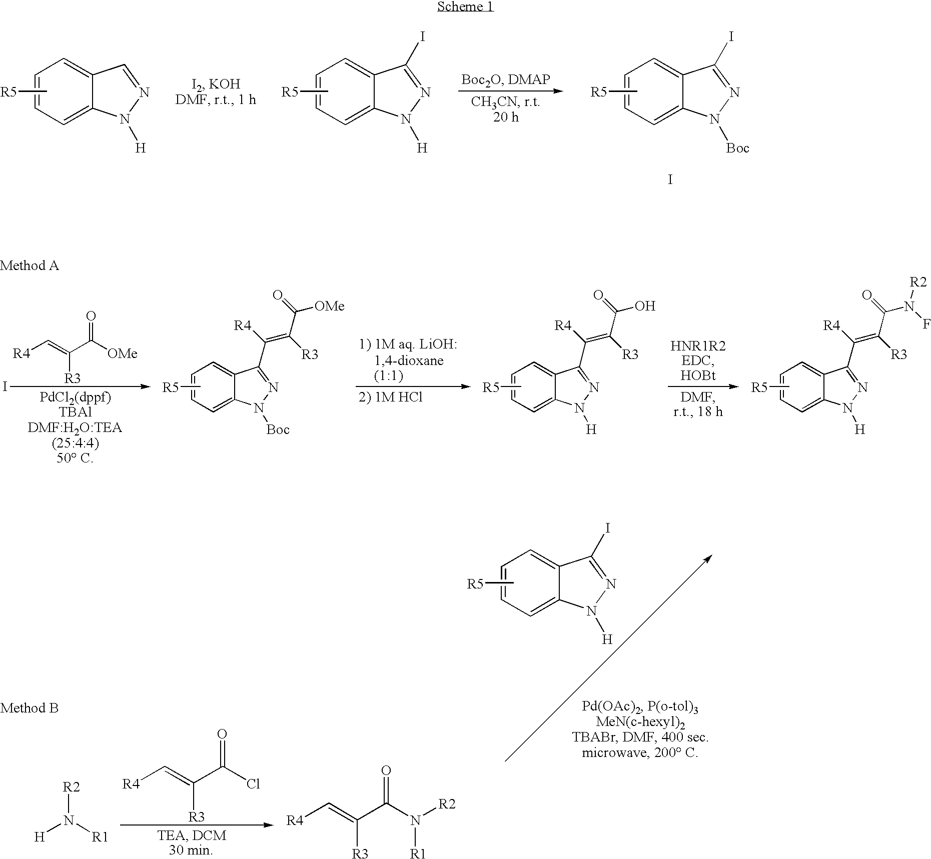

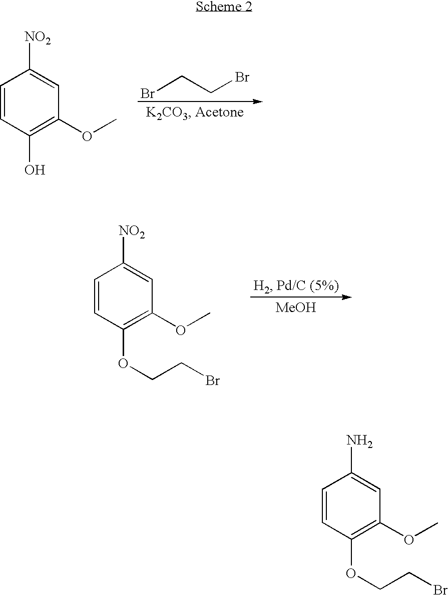

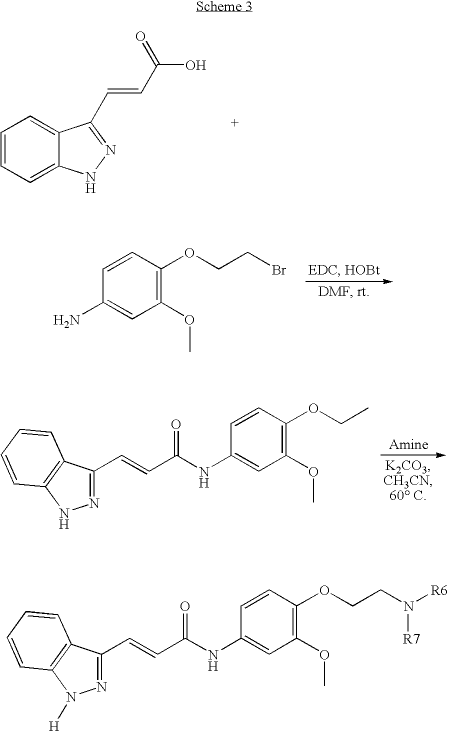

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megahertz); | mol (moles); |
| mmol (millimoles); | rt (room temperature); |
| min (minutes); | h (hours); |
| mp (melting point); | TLC (thin layer |
| | chromatography); |
| Tᵣ (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N- |
| dimethylformamide); | |
| DMPU (N,N′-dimethylpropyleneurea); | CDI (1,1- |
| | carbonyldiimidazole); |
| IBCF (isobutyl chloroformate); | HOAc (acetic |
| acid); | |
| HOSu (N-hydroxysuccinimide); | HOBT (1- |
| | hydroxybenzotriazole); |
| mCPBA (meta-chloroperbenzoic acid; | |
| EDC (1-[3-dimethylamino) propyl]- | |
| 3-ethylcarbodiimide hydrochloride); | |
| BOC (tert-butyloxycarbonyl); | FMOC (9- |
| | fluorenylmethoxycarbonyl); |
| DCC (dicyclohexylcarbodiimide); | CBZ (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | BSA (bovine serum albumin) |
| ATP (adenosine triphosphate); | HRP (horseradish peroxidase); |
| DMEM (Dulbecco's modified | |
| Eagle medium); | |
| HPLC (high pressure liquid | |
| chromatography); | |
| BOP (bis(2-oxo-3- | |
| oxazolidinyl)phosphinic chloride); | |
| TBAF (tetra-n-butylammonium fluoride); | |
| HBTU(O-Benzotriazole-1-yl-N,N,N′,N′- | |
| tetramethyluroniumhexafluoro phosphate). | |
| HEPES (4-(2-hydroxyethyl)-1-piperazine | |
| ethane sulfonic acid); | |
| DPPA (diphenylphosphoryl azide); | |
| fHNO₃ (fuming HNO₃); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

HPLC were recorded on a Gilson HPLC or Shimazu HPLC system by the following conditions. Column: 50×4.6 mm (id) stainless steel packed with 5 μm Phenomenex Luna C-18; Flow rate: 2.0 mL/min; Mobile phase: A phase=50 mM ammonium acetate (pH 7.4), B phase=acetonitrile, 0-0.5 min (A: 100%, B: 0%), 0.5-3.0 min (A:100-0%, B:0-100%), 3.0-3.5 min (A: 0%, B: 100%), 3.5-3.7 min (A: 0-100%, B: 100-0%), 3.7-4.5 min (A: 100%, B: 0%); Detection: UV 254 nm; Injection volume: 3 μL.

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Intermediate Example 1

### E)-3-(1H-indazol-3-yl)-2-propenoic acid

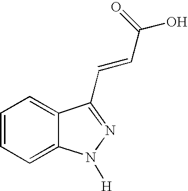

Methyl (2E)-3-(1H-indazol-3-yl)-2-propenoate (See Scheme 1, Method A—0.500 g, 1.65 mmol) was dissolved in 1,4-dioxane (5.5 mL) with magnetic stirring. 1M aq. Lithium hydroxide (5.5 mL) was then added to the solution and the reaction was stirred at ambient temperature for 4 hours. Next, 1M aq. hydrochloric acid (11 mL) was added to the reaction and the stirring continued for 20 minutes. The solids were collected by suction filtration and dried to give (2E)-3-(1H-indazol-3-yl)-2-propenoic acid (0.218 g) as a white solid. ¹H NMR (DMSO-d₆): δ 13.62 (br s, 1H), 12.47 (br s, 1H), 8.09 (d, 1H), 7.86 (d, 1H), 7.64 (d, 1H), 7.46 (dd, 1H), 7.27 (dd, 1H), 6.72 (d, 1H). MS m/z 187 (M−1).

### Intermediate Example 2

### N,N-diisopropyl-N-[2-(2-methoxy-4-nitrophenoxy)ethyl]amine

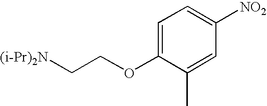

To a magnetically stirred solution of 4-nitroguaiacol (3.63 g, 21.5 mmol) in acetone (50 mL) was added N-(2-chloroethyl)-N,N-diisopropylamine hydrochloride (4.73 g, 23.6 mmol) and then potassium carbonate (6.25 g, 45.2 mmol). The reaction was stirred at ambient temperature for 18 hours overnight. The reaction was poured into water (100 mL) and the mixture was extracted with diethyl ether (2×100 mL). The combined organic extract was washed with water (50 mL), saturated brine (50 mL), and then dried (MgSO4) for 3 hours. The mixture was filtered and the filtrate was concentrated to dryness to give N,N-diisopropyl-N-[2-(2-methoxy-4-nitrophenoxy)-ethyl]amine (5.37 g) of a waxy yellow solid. ¹H NMR (DMSO-d₆): δ 7.86 (d, 1H), 7.71 (s, 1H), 7.16 (d, 1H), 3.99 (t, 2H), 3.86 (s, 3H), 2.99 (m, 2H), 2.78 (t, 2H), 0.96 (d, 12H). MS m/z 297 (M+1).

### Intermediate Example 3

### N-[2-(4-amino-2-methoxyphenoxy)ethyl]-N,N-diisopropylamine

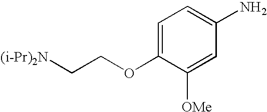

To a solution of N,N-diisopropyl-N-[2-(2-methoxy-4-nitrophenoxy)ethyl]amine (Intermediate Example 2—5.36 g, 18.1 mmol) in ethanol (100 mL) was added a slurry of 10% palladium on carbon (1.07 g) in water (5 mL) under a blanket of nitrogen. A hydrogen filled balloon was connected to the reaction flask and the reaction was magnetically stirred overnight for 18 hours. The reaction mixture was filtered through Celite and the pad was rinsed with ethanol. The combined filtrate was concentrated to dryness to give N-[2-(4-amino-2-methoxy-phenoxy)ethyl]-N,N-diisopropylamine (4.65 g) as a dark oil. ¹H NMR (DMSO-d₆): δ 6.59 (d, 1H), 6.22 (s, 1H), 6.01 (d, 1H), 4.64 (br s, 2H), 3.65 (t, 2H), 3.63 (s, 3H), 2.94 (m, 2H), 2.64 (t, 2H), 0.93 (d, 12H). MS m/z 267 (M+1).

### Intermediate Example 4

### N-{4-[2-(diisopropylamino)ethoxy]-3-methoxyphenyl}acrylamide

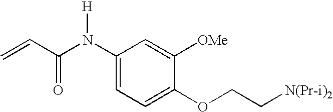

To a solution of N-[2-(4-amino-2-methoxyphenoxy)ethyl]-N,N-diisopropylamine (Intermediate Example 3—0.286 g, 1.07 mmol) in dichloromethane (2.0 mL) was added dropwise a solution of acryloyl chloride (87.2 μL, 1.07 mmol) in dichloromethane (1.0 mL) at 0° C. The ice bath was removed and the reaction was stirred for 2 hours at ambient temperature. The reaction was diluted with dichloromethane (15 mL) and water (3 mL) was added with stirring. The phases were separated and the organic layer was washed with saturated brine (3 mL) and dried with MgSO₄. The mixture was filtered and the filtrate was concentrated under reduced pressure to give N-{4-[2-(diisopropylamino)ethoxy]-3-methoxyphenyl}-acrylamide (0.266 g) as a dark oil. ¹H NMR (DMSO-d₆): δ 9.98 (br s, 1H), 7.35 (s, 1H), 7.12 (d, 1H), 6.87 (d, 1H), 6.37 (dd, 1H), 6.19 (d, 1H), 5.70 (d, 1H), 3.78 (t, 2H), 3.71 (s, 3H), 2.97 (m, 2H), 2.71 (t, 2H), 0.95 (d, 12H). MS m/z 321 (M+1).

### Intermediate Example 5

### 4-(2-bromoethoxy)-3-methoxyaniline

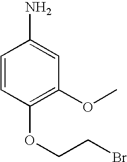

A mixture of 1-(2-bromoethoxy)-2-methoxy-4-nitrobenzene (2.35 g, 8.5 mmol) and 0.30 g of 5% Pd/C in 300 ml MeOH was hydrogenated at room temperature, using a hydrogen filled balloon for 1.5 hrs. The reaction was monitored by TLC. After the reaction was completed, the mixture was filtered, and the filtrate was evaporated at room temperature to give 4-(2-bromoethoxy)-3-methoxyaniline (1.95 g) as a brown oil. This compound was not stable when stored at room temperature and was used in the next reaction immediately. ¹H NMR (CDCl₃): δ 6.78 (d, 1H), 6.42 (s, 1H), 6.32 (d, 1H), 4.22 (t, 2H), 3.80 (s, 3H), 3.59 (t, 2H). MS m/z 246 (M+1).

### Intermediate Example 6

### E)-N-[4-(2-bromoethoxy)-3-methoxyphenyl]-3-(1H-indazol-3-yl)-2-propenamide

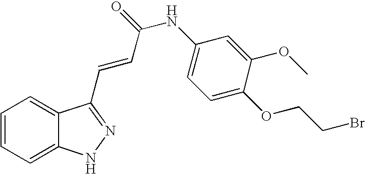

To a magnetically stirred solution of (2E)-3-(1-H-indazol-3-yl)-2-propenoic acid (Intermediate Example 1—1.60 g, 8.5 mmol), 1-[3-(Dimethylamino)propyl]-3-ethylcarbodiimide hydrochloride (EDC) (1.65 g, 8.60 mmol) and 1-hydroxybenzotriazole (HOBT) (1.16 g, 8.6 mmol) in 30 ml DMF was added 4-(2-bromoethoxy)-3-methoxyaniline (Intermediate Example 5—2.11 g, 8.6 mmol) solution in 30 ml DMF. After the reaction was stirred at room temperature overnight, 400 ml water was added to the reaction mixture. The brown precipitate was filtered and washed with hexane/ether (1:1) to give (2E)-N-[4-(2-bromoethoxy)-3-methoxyphenyl]-3-(1H-indazol-3-yl)-2-propenamide 2.2 g as a brown solid. ¹H NMR (DMSO-d₆): δ 13.5 (s, 1H), 10.1 (s, 1H), 8.06 (d, 1H), 7.76 (d, 1H), 7.60 (d, 1H), 7.48 (s, 1H), 7.43 (t, 1H), 7.28 (t, 1H), 7.18 (s, 1H), 7.15 (d, 1H), 6.95 (d, 1H), 4.25 (t, 2H), 3.77 (s, 3H), 3.75 (t, 2H). MS m/z 416 (M+1).

### Example 1

### (2E)-N-(1,3-benzothiazol-6-yl)-3-(1H-indazol-3-yl)-2-propenamide

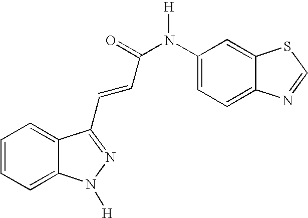

A solution of acryloyl chloride (12.9 μL, 0.159 mmol) in dichloromethane (1.0 mL) was added to 1,3-benzothiazol-6-amine (23.9 mg, 0.159 mmol) in a 0.5-2 mL conical reaction vial fitted with rubber-lined aluminum septum-cap. Triethylamine (22.2 μL, 0.159 mmol) was added to the reaction mixture and the mixture was stirred for 1 hour at ambient temperature. The reaction mixture was concentrated to dryness under reduced pressure and the residue was dissolved in dimethylformamide (0.5 mL). To the residue was added a stock solution containing 3-iodoindazole (35.4 mg, 0.145 mmol), palladium (II) acetate (1.4 mg, 0.0058 mmol), tri-o-tolylphosphine (4.4 mg, 0.014 mmol), tetrabutylammonium bromide (46.7 mg, 0.145 mmol), N-methyl dicyclohexylamine (37.3 μL, 0.174 mmol), and dimethylformamide (2.0 mL). The sealed reaction vial was microwaved at 200° C. for 400 seconds in a Personal Chemistry SmithSynthesizer®. After cooling, the reaction vial cap was removed and silica gel 60 (40-63μ; 1.5 g) was added to the reaction mixture. The volatiles were removed by heating in a Savant Speed-vac Plus for 18 hours under high vacuum. The pre-adsorbed silica gel was packed into a solid loading cartridge (ISCO) and eluted onto a pre-packed Redi-sep silica gel column (4.2 g; ISCO) using a gradient elution EtOAc:hexanes (50:50) to EtOAc (100%) over 8 minutes, followed by elution with EtOAc for 5 minutes. The appropriate fractions containing product (based on LC-MS analysis) were combined and concentrated to dryness. Trituration of the residue with ether/hexanes (0.5 mL/0.5 mL), collection of the solids by filtration, and drying under vacuum at 60° C. for 2 hours gave (2E)-N-(1,3-benzothiazol-6-yl)-3-(1H-indazol-3-yl)-2-propenamide (23 mg) as a beige solid. ¹H NMR (DMSO-d₆): δ 13.56 (br s, 1H), 10.54 (br s, 1H), 9.29 (s, 1H), 8.72 (s, 1H), 8.11 (d, 1H), 8.06 (d, 1H), 7.86 (d, 1H), 7.68 (d, 1H), 7.63 (d, 1H), 7.46 (dd, 1H), 7.32 (d, 1H), 7.26 (d, 1H). MS m/z 321 (M+1).

### Example 2

### (2E)-N-(3,4-dimethyl-5-isoxazolyl)-3-(1H-indazol-3-yl)-2-propenamide

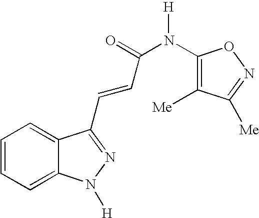

Utilizing the procedure described in Example 1 and in Scheme 1, method B, 3,4-dimethyl-5-isoxazolamine (17.8 mg, 0.159 mmol) gave (2E)-N-(3,4-dimethyl-5-isoxazolyl)-3-(1H-indazol-3-yl)-2-propenamide (8.6 mg) as a light amber semi-solid. ¹H NMR (DMSO-d₆): δ 13.58 (br s, 1H), 10.75 (br s, 1H), 8.03 (d, 1H), 7.83 (d, 1H), 7.60 (d, 1H), 7.42 (dd, 1H), 7.27 (dd, 1H), 7.14 (d, 1H), 2.14 (s, 3H), 1.85 (s, 3H). MS m/z 283 (M+1).

### Example 3

### (2E)-N-(2-cyanophenyl)-3-(1H-indazol-3-yl)-2-propenamide

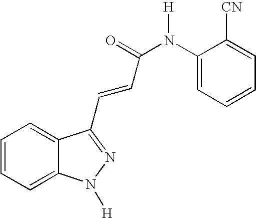

Utilizing the procedure described in Example 1 and in Scheme 1, method B, 2-aminobenzonitrile (18.8 mg, 0.159 mmol) gave (2E)-N-(2-cyanophenyl)-3-(1H-indazol-3-yl)-2-propenamide (13 mg) as a pale yellow solid. ¹H NMR (DMSO-d₆): δ 13.59 (br s, 1H), 10.43 (br s, 1H), 8.14 (d, 1H), 7.87 (d, 1H), 7.85 (d, 1H), 7.83 (d, 1H), 7.73 (dd, 1H), 7.64 (d, 1H), 7.46 (dd, 1H), 7.36 (dd, 1H), 7.34 (d, 1H), 7.31 (dd, 1H). MS m/z 289 (M+1).

### Example 4

### (2E)-3-(1H-indazol-3-yl)-N-(6-methoxy-3-pyridinyl)-2-propenamide

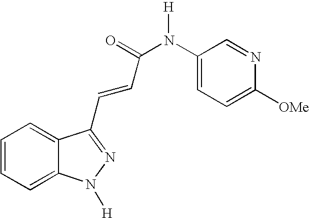

Utilizing the procedure described in Example 1 and in Scheme 1, method B, 6-methoxy-3-pyridinamine (19.7 mg, 0.159 mmol) gave (2E)-3-(1H-indazol-3-yl)-N-(6-methoxy-3-pyridinyl)-2-propenamide (8.7 mg) as a rust brown solid. ¹H NMR (DMSO-d₆): δ 13.51 (br s, 1H), 10.27 (br s, 1H), 8.46 (s, 1H), 8.05 (d, 1H), 8.01 (dd, 1H), 7.78 (d, 1H), 7.59 (d, 1H), 7.42 (dd, 1H), 7.27 (dd, 1H), 7.15 (d, 1H), 6.82 (d, 1H), 3.81 (s, 3H). MS m/z 295 (M+1).

### Example 5

### (2E)-N-(3-chlorophenyl)-3-(1H-indazol-3-yl)-2-propenamide

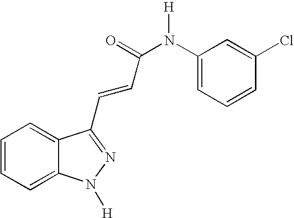

Utilizing the procedure described in Example 1 and in Scheme 1, method B, 1,3-chloroaniline (20.3 mg, 0.159 mmol) gave (2E)-N-(3-chlorophenyl)-3-(1H-indazol-3-yl)-2-propenamide (9.0 mg) as an off-white solid. ¹H NMR (DMSO-d₆): δ 13.57 (br s, 1H), 10.44 (br s, 1H), 8.08 (d, 1H), 7.98 (d, 1H), 7.84 (d, 1H), 7.62 (d, 1H), 7.52 (d, 1H), 7.46 (dd, 1H), 7.39 (dd, 1H), 7.31 (dd, 1H), 7.18 (d, 1H), 7.14 (d, 1H). MS m/z 298 (M+1).

### Example 6

### (2E)-N-(2,3-dihydro-1H-inden-5-yl)-3-(1H-indazol-3-yl)-2-propenamide

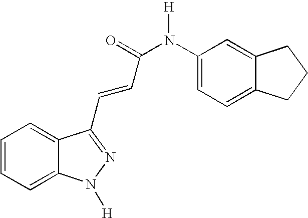

Utilizing the procedure described in Example 1 and in Scheme 1, method B, 2,3-dihydro-1H-inden-5-ylamine (21.2 mg, 0.159 mmol) gave (2E)-N-(2,3-dihydro-1H-inden-5-yl)-3-(1H-indazol-3-yl)-2-propenamide (15 mg) as a pale beige solid. ¹H NMR (DMSO-d₆): δ 13.50 (br s, 1H), 10.13 (br s, 1H), 8.09 (d, 1H), 7.78 (d, 1H), 7.66 (s, 1H), 7.62 (d, 1H), 7.46 (d, 1H), 7.42 (dd, 1H), 7.29 (dd, 1H), 7.20 (d, 1H), 7.18 (d, 1H), 2.85 (m, 4H), 2.02 (m, 2H). MS m/z 304 (M+1).

### Example 7

### (2E)-N-[4-(dimethylamino)phenyl]-3-(1H-indazol-3-yl)-2-propenamide

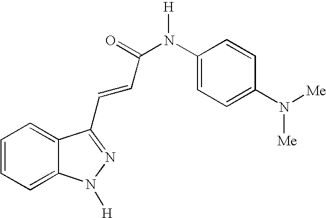

Utilizing the procedure described in Example 1 and in Scheme 1, method B, N¹,N¹-dimethyl-1,4-benzenediamine (21.7 mg, 0.159 mmol) gave (2E)-N-[4-(dimethylamino)phenyl]-3-(1H-indazol-3-yl)-2-propenamide (1.8 mg) as a dark brown solid. MS m/z 307 (M+1).

### Example 8

### (2E)-N-(3-cyclopropyl-1-methyl-1H-pyrazol-5-yl)-3-(1H-indazol-3-yl)-2-propenamide

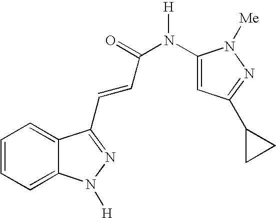

Utilizing the procedure described in Example 1 and in Scheme 1, method B, 3-cyclopropyl-1-methyl-1H-pyrazol-5-ylamine (21.8 mg, 0.159 mmol) gave (2E)-N-(3-cyclopropyl-1-methyl-1H-pyrazol-5-yl)-3-(1H-indazol-3-yl)-2-propenamide (19 mg) as a light amber semi-solid. ¹H NMR (DMSO-d₆): δ 13.54 (br s, 1H), 10.10 (br s, 1H), 8.06 (d, 1H), 7.79 (d, 1H), 7.59 (d, 1H), 7.42 (dd, 1H), 7.27 (dd, 1H), 7.23 (d, 1H), 6.06 (s, 1H), 3.62 (s, 3H), 1.93 (m, 1H), 0.79 (m, 2H), 0.59 (m, 2H). MS m/z 308 (M+1).

### Example 9

### (2E)-3-(1H-indazol-3-yl)-N-(5-quinolinyl)-2-propenamide

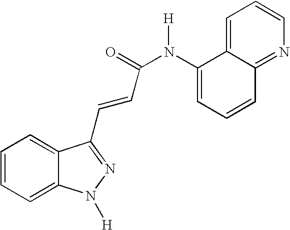

Utilizing the procedure described in Example 1 and in Scheme 1, method B, 5-quinolinamine (22.9 mg, 0.159 mmol) gave (2E)-3-(1H-indazol-3-yl)-N-(5-quinolinyl)-2-propenamide (4.9 mg) as a light brown solid. ¹H NMR (DMSO-d₆): δ 13.56 (br s, 1H), 10.33 (br s, 1H), 8.95 (d, 1H), 8.65 (d, 1H), 8.18 (d, 1H), 8.09 (d, 1H), 7.88 (d, 1H), 7.87 (d, 1H), 7.79 (dd, 1H), 7.63 (2×dd, 2H), 7.50 (d, 1H), 7.48 (d, 1H), 7.32 (dd, 1H). MS m/z 315 (M+1).

### Example 10

### (2E)-N-[3-(acetylamino)phenyl]-3-(1H-indazol-3-yl)-2-propenamide

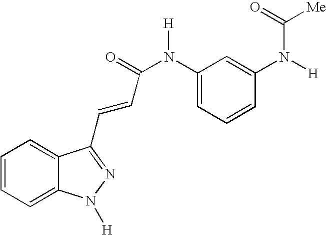

Utilizing the procedure described in Example 1 and in Scheme 1, method B, N-(3-aminophenyl)acetamide (23.9 mg, 0.159 mmol) gave (2E)-N-[3-(acetylamino)phenyl]-3-(1H-indazol-3-yl)-2-propenamide (9.1 mg) as an ocher solid. ¹H NMR (DMSO-d₆): δ 13.53 (br s, 1H), 10.25 (br s, 1H), 9.99 (br s, 1H), 8.10 (d, 1H), 8.05 (s, 1H), 7.80 (d, 1H), 7.62 (d, 1H), 7.48 (m, 2H), 7.32 (d, 1H), 7.25 (m, 3H), 2.05 (s, 3H). MS m/z 321 (M+1).

### Example 11

### (2E)-3-(1H-indazol-3-yl)-N-(3,4,5-trimethoxyphenyl)-2-propenamide

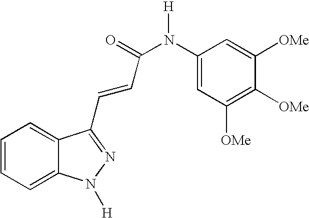

Utilizing the procedure described in Example 1 and in Scheme 1, method B, 3,4,5-trimethoxyaniline (29.1 mg, 0.159 mmol) gave (2E)-3-(1H-indazol-3-yl)-N-(3,4,5-trimethoxyphenyl)-2-propenamide (21 mg) as a beige solid. ¹H NMR (DMSO-d₆): δ 13.53 (br s, 1H), 10.21 (br s, 1H), 8.08 (d, 1H), 7.79 (d, 1H), 7.62 (d, 1H), 7.46 (dd, 1H), 7.30 (dd, 1H), 7.18 (d, 1H), 7.11 (s, 2H), 3.78 (s, 6H), 3.64 (s, 3H). MS m/z 354 (M+1).

### Example 12

### (2E)-N-(3-benzoylphenyl)-3-(1H-indazol-3-yl)-2-propenamide

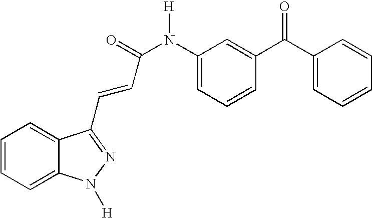

Utilizing the procedure described in Example 1 and in Scheme 1, method B, (3-aminophenyl)(phenyl)methanone (31.4 mg, 0.159 mmol) gave (2E)-N-(3-benzoylphenyl)-3-(1H-indazol-3-yl)-2-propenamide (15 mg) as a tan solid. ¹H NMR (DMSO-d₆): δ 13.55 (br s, 1H), 10.50 (br s, 1H), 8.08 (m, 3H), 7.82 (d, 1H), 7.78 (m, 2H), 7.70 (d, 1H), 7.59 (m, 4H), 7.45 (m, 2H), 7.30 (dd, 1H), 7.20 (d, 1H). MS m/z 368 (M+1).

### Example 13

### (2E)-N-[3-chloro-4-(4-morpholinyl)phenyl]-3-(1H-indazol-3-yl)-2-propenamide

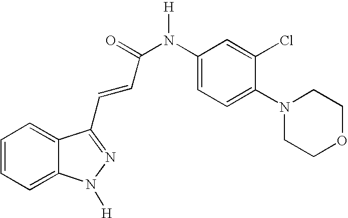

Utilizing the procedure described in Example 1 and in Scheme 1, method B, 3-chloro-4-(4-morpholinyl)aniline (33.8 mg, 0.159 mmol) gave (2E)-N-[3-chloro-4-(4-morpholinyl)phenyl]-3-(1H-indazol-3-yl)-2-propenamide (17 mg) as a beige solid. ¹H NMR (DMSO-d₆): δ 13.54 (br s, 1H), 10.32 (br s, 1H), 8.08 (d, 1H), 7.96 (s, 1H), 7.80 (d, 1H), 7.62 (d, 1H), 7.53 (d, 1H), 7.45 (dd, 1H), 7.30 (dd, 1H), 7.18 (d, 1H), 7.16 (d, 1H), 3.74 (m, 4H), 2.94 (m, 4H). MS m/z 383 (M+1).

### Example 14

### (2E)-N-{5-[(diethylamino)sulfonyl]-2-methoxyphenyl}-3-(1H-indazol-3-yl)-2-propenamide

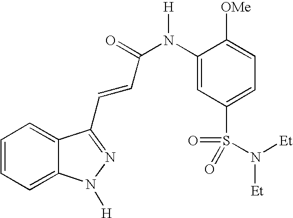

Utilizing the procedure described in Example 1 and in Scheme 1, method B, 3-amino-N,N-diethyl-4-methoxy-benzenesulfonamide (41.1 mg, 0.159 mmol) gave (2E)-{5-[(diethylamino)sulfonyl]-2-methoxyphenyl}-3-(1H-indazol-3-yl)-2-propenamide (32 mg) as a light amber oil. ¹H NMR (DMSO-d₆): δ 13.51 (br s, 1H), 9.81 (br s, 1H), 8.78 (s, 1H), 8.24 (d, 1H), 7.79 (d, 1H), 7.57 (m, 3H), 7.43 (dd, 1H), 7.28 (dd, 1H), 7.23 (d, 1H), 3.97 (s, 3H), 3.13 (m, 4H), 1.04 (m, 6H). MS m/z 429 (M+1).

### Example 15

### (2E)-N-[4-[2-(diisopropylamino)ethoxy]-3-methoxyphenyl]-3-(1H-indazol-3-yl)-2-propenamide

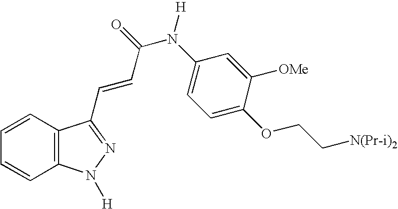

To a solution of (2E)-3-(1H-indazol-3-yl)-2-propenoic acid (Intermediate Example 1—0.200 g, 1.06 mmol) in dimethylformamide (18 mL) was added polymer supported carbodiimide resin (1.85 g, 1.77 mmol; loading=0.96 mmol/g) and 1-hydroxybenzotriazole (0.180 g, 1.33 mmol). After 30 minutes of stirring a solution of N-[2-(4-amino-2-methoxyphenoxy)ethyl]-N,N-diisopropylamine (Intermediate Example 3—0.236 g, 0.886 mmol) in dimethylformamide (2 mL) was added to the reaction mixture which was stirred for 64 hours. The reaction mixture was filtered to remove the resin and the resin was washed with dimethylformamide (10 mL). The combined filtrate was treated with MP-carbonate resin (1.50 g, 4.29 mmol; loading=2.86 mmol/g) and the mixture was stirred for 18 hours. The reaction mixture was filtered to remove resin and the resin was washed with dimethylformamide (5 mL). The combined filtrate was concentrated to a dark oil. The oil was dissolved in ethyl acetate (5 mL) and the solution was applied to a Redi-sep silica gel cartridge (35 g; ISCO) and the column was eluted with ethyl acetate, then with methanol:ethyl acetate (10:90). Fractions containing product were combined and concentrated to dryness under reduced pressure. The resulting semi-solid was treated with hexanes:ether (75 mL:5 mL) and the mixture was magnetically stirred vigorously for 64 hours. The solids were collected by filtration and rinsed with ether (1-2 mL) followed by drying to give (2%)-N-{4-[2-(diisopropyl-amino)ethoxy]-3-methoxyphenyl}-3-(1H-indazol-3-yl)-2-propenamide (0.093 g) as a pale yellow solid. ¹H NMR (DMSO-d₆): δ 13.49 (br s, 1H), 10.11 (br s, 1H), 8.06 (d, 1H), 7.75 (d, 1H), 7.60 (d, 1H), 7.43 (m, 2H), 7.27 (dd, 1H), 7.16 (d, 1H), 7.15 (s, 1H), 6.91 (d, 1H), 3.80 (t, 2H), 3.74 (s, 3H), 2.98 (m, 2H), 2.72 (t, 2H), 0.96 (d, 12H). MS m/z 437 (M+1).

### Example 16

### (2E)-3-(1H-indazol-3-yl)-N-(3-methoxy-4-{2-[(2-phenoxyethyl)amino]ethoxy}phenyl)-2-propenamide

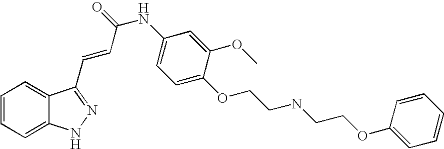

To a magnetically stirred solution of (2E)-N-[4-(2-bromoethoxy)-3-methoxyphenyl]-3-(1H-indazol-3-yl)-2-propenamide (Intermediate Example 6—40 mg, 0.10 mmol) in acetonitrile (2.0 mL) was added 2-phenoxyethanamine (28 mg, 0.20 mmol) and potassium carbonate (53 mg, 0.40 mmol). The reaction mixture was heated at 60° C. overnight. After the reaction was cooled down, the mixture was filtered through a pad of Celite. The solvent was evaporated. The residue was purified by chromatography (10% methanol in ethyl acetate) to give (2E)-3-(1H-indazol-3-yl)-N-(3-methoxy-4-{2-[(2-phenoxyethyl)-amino]ethoxy}phenyl)-2-propenamide (15 mg) as a yellow solid. ¹H NMR (CD3OD): δ 8.09 (d, 1H), 8.00 (d, 1H), 7.62-7.56 (m, 2H), 7.48 (t, 1H), 7.34-7.15 (m, 5H), 7.01-6.93 (m, 4H), 4.15 (2t, 4H), 3.89 (s, 3H), 3.10 (2t, 4H). MS m/z 473 (M+1).

### Example 17

### (2E)-3-(1H-indazol-3-yl)-N-[3-methoxy-4-[2-(4-morpholinyl)ethoxy]phenyl]-2-propenamide

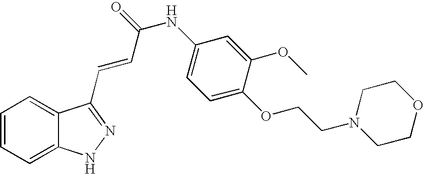

Utilizing the procedure of Example 16 and Schemes 2 and 3, morpholine (17 mg, 0.20 mmol) gave (2E)-3-(1H-indazol-3-yl)-N-{3-methoxy-4-[2-(4-morpholinyl)ethoxy]phenyl}-2-propenamide (20 mg) as a yellow solid. ¹H NMR (CD3OD): δ 8.04 (d, 1H), 7.94 (d, 1H), 7.56 (d, 1H), 7.50 (s, 1H), 7.44 (t, 1H), 7.27 (t, 1H), 7.15-7.11 (m, 2H), 6.93 (d, 1H), 4.11 (t, 2H), 3.84 (s, 3H), 3.70 (m, 4H), 2.78 (t, 2H), 2.61 (m, 4H). MS m/z 423 (M+1).

### Example 18

### E)-3-(1H-indazol-3-yl)-N-(3-methoxy-4-{2-[(2-methoxyethyl)amino]-ethoxy}phenyl)-2-propenamide

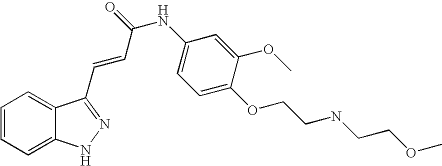

Utilizing the procedure of Example 16 and Schemes 2 and 3, 2-methoxy ethanamine (15 mg, 0.20 mmol) gave (2E)-3-(1H-indazol-3-yl)-N-(3-methoxy-4-{2-[(2-methoxyethyl)amino]ethoxy}phenyl)-2-propenamide (13 mg) as a yellow solid. ¹H NMR (CD3OD): δ 8.09 (d, 1H), 7.99 (d, 1H), 7.63-7.56 (m, 2H), 7.49 (t, 1H), 7.32 (t, 1H), 7.20-7.15 (m, 2H), 6.99 (d, 1H), 4.13 (t, 2H), 3.90 (s, 3H), 3.57 (t, 2H), 3.40 (s, 3H), 2.99 (t, 2H), 2.87 (t, 2H). MS m/z 411 (M+1).

### Example 19

### (2E)-3-(1H-indazol-3-yl)-N-(3-methoxy-4-{2-[methyl(propyl)amino]ethoxy}phenyl)-2-propenamide

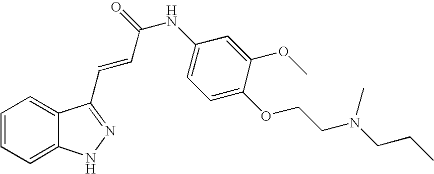

Utilizing the procedure of Example 16 and Schemes 2 and 3, N-methyl-N-propylamine (15 mg, 0.20 mmol) gave (2E)-3-(1H-indazol-3-yl)-N-(3-methoxy-4-{2-[methyl(propyl)amino]ethoxy}phenyl)-2-propenamide (25 mg) as a yellow solid. ¹H NMR (CD3OD): δ 8.10 (d, 1H), 7.99 (d, 1H), 7.63-7.56 (m, 2H), 7.49 (t, 1H), 7.32 (t, 1H), 7.23-7.18 (m, 2H), 6.99 (d, 1H), 4.17 (t, 2H), 3.90 (s, 3H), 2.99 (t, 2H), 2.63 (t, 2H), 2.50 (s, 3H), 1.63 (q, 2H), 0.98 (t, 3H). MS m/z 409 (M+1).

### Example 20

### (2E)-N-[4-(2-{[2-[(4-chlorophenyl)ethyl]amino}ethoxy)-3-methoxyphenyl]-3-(1H-indazol-3-yl)-2-propenamide

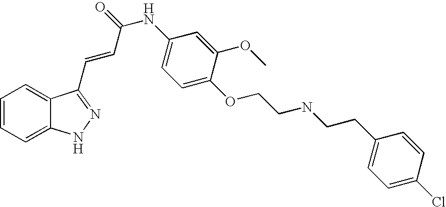

Utilizing the procedure of Example 16 and Schemes 2 and 3,2-(4-chlorophenyl)ethanamine (31 mg, 0.20 mmol) gave (2E)-N-[4-(2-{[2-(4-chlorophenyl)ethyl]amino}ethoxy)-3-methoxyphenyl]-3-(1H-indazol-3-yl)-2-propenamide (17 mg) as a yellow solid. ¹H NMR (CD3OD): δ 8.09 (d, 1H), 8.00 (d, 1H), 7.62 (d, 1H), 7.55 (s, 1H), 7.48 (t, 1H), 7.33-7.15 (m, 7H), 6.94 (d, 1H), 4.12 (t, 2H), 3.96 (s, 3H), 3.04 (t, 2H), 2.95 (d, 2H), 2.85 (d, 2H). MS m/z 491 (M+1).

### Example 21

### Ethyl 4-{[2-(4-{[(2E)-3-(1H-indazol-3-yl)-2-propenoyl]amino}-2-methoxyphenoxy)ethyl]amino}-1-piperidine carboxylate

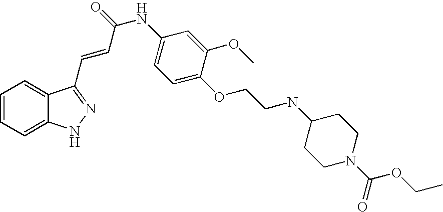

Utilizing the procedure of Example 16 and Schemes 2 and 3, ethyl 4-amino-1-piperidine carboxylate (35 mg, 0.20 mmol) gave ethyl 4-{[2-(4-{[(2E)-3-(1H-indazol-3-yl)-2-propenoyl]amino}-2-methoxyphenoxy)ethyl]amino}-1-piperidine carboxylate (23 mg) as a yellow solid. ¹H NMR (CD3OD): δ 8.09 (d, 1H), 8.00 (d, 1H), 7.63-7.57 (m, 2H), 7.48 (t, 1H), 7.32 (t, 1H), 7.18 (2d, 2H), 6.98 (d, 1H), 4.92 (bs, 6H), 3.91 (s, 3H), 3.04 (bs, 2H), 2.88-2.82 (m, 3H), 1.97 (d, 2H), 1.28 (bs, 5H). MS m/z 508 (M+1).

### Example 22

### (2E)-N-[4-[2-(4-acetyl-1-piperazinyl)ethoxy]-3-methoxyphenyl]-3-(1H-indazol-3-yl)-2-propenamide

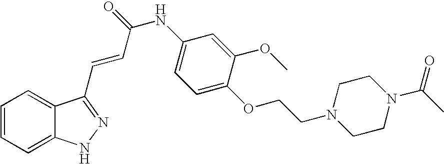

Utilizing the procedure of Example 16 and Schemes 2 and 3,1-acetyl piperazine (26 mg, 0.20 mmol) gave (2E)-N-{4-[2-(4-acetyl-1-piperazinyl)ethoxy]-3-methoxyphenyl}-3-(1H-indazol-3-yl)-2-propenamide (32 mg) as a yellow solid. ¹H NMR (CD3OD): δ 8.10 (d, 1H), 8.04 (d, 1H), 7.62 (d, 1H), 7.56 (s, 1H), 7.49 (t, 1H), 7.33 (t, 1H), 7.23-7.18 (m, 2H), 7.00 (d, 1H), 4.17 (t, 2H), 3.90 (s, 3H), 3.66-3.58 (m, 4H), 2.87 (t, 2H), 2.71-2.61 (m, 4H), 2.13 (s, 3H). MS m/z 464 (M+1).

### Example 23

### (2E)-N-benzyl-3-(1H-indazol-3-yl)prop-2-enamide

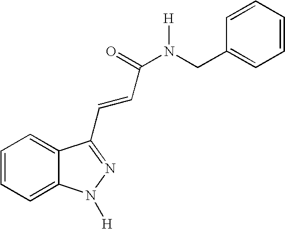

Utilizing the procedure described in Example 1 and in Scheme 1, method B, benzylamine (61.5 mg, 0.574 mmol) gave (2E)-N-benzyl-3-(1H-indazol-3-yl)prop-2-enamide (30 mg) as a white solid. ¹H NMR (DMSO-d₆) δ 13.43 (s, 1H) 8.64 (t, J=5.86 Hz, 1H) 8.02 (d, J=8.24 Hz, 1H) 7.69 (d, J=16.11 Hz, 1H) 7.58 (d, J=8.42 Hz, 1H) 7.41 (m, 1H) 7.34 (m, 4H) 7.25 (m, 2H) 7.07 (d, J=16.11 Hz, 1H) 4.43 (d, J=5.86 Hz, 2H). MS m/z 278 (M+1).

### Example 24

### (2E)-3-(1H-indazol-3-yl)-N-isobutylprop-2-enamide

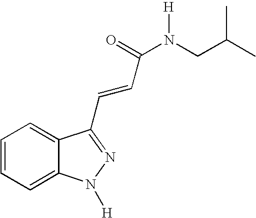

Utilizing the procedure described in Example 1 and in Scheme 1, method B, isobutylamine (41.9 mg, 0.574 mmol) gave (2E)-3-(1H-indazol-3-yl)-N-isobutylprop-2-enamide (20 mg) as a white solid. ¹H NMR (DMSO-d₆) δ 13.40 (s, 1H) 8.13 (t, J=5.58 Hz, 1H) 8.03 (d, J=8.24 Hz, 1H) 7.63 (d, J=16.11 Hz, 1H) 7.58 (d, J=8.79 Hz, 1H) 7.41 (t, J=7.69 Hz, 1H) 7.24 (t, J=7.69 Hz, 1H) 7.05 (d, J=16.30 Hz, 1H) 3.04 (t, J=6.32 Hz, 2H) 1.75 (m, 1H) 0.89 (d, J=6.59 Hz, 6H). MS m/z 244 (M+1).

### Example 25

### (2E)-3-(1H-indazol-3-yl)-N-(3-morpholin-4-ylpropyl)prop-2-enamide hydrochloride

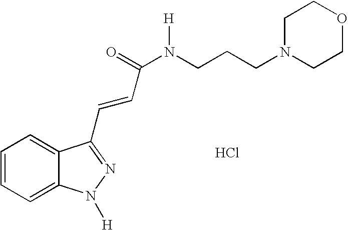

Utilizing the procedure described in Example 1 and in Scheme 1, method B, (except after chromatography, conc. HCl was added to generate the hydrochloride salt), N-(3-aminopropyl)morpholine (82.7 mg, 0.574 mmol) gave (2E)-3-(1H-indazol-3-yl)−/−(3-morpholin-4-ylpropyl)-prop-2-enamide HCl (34 mg) as a tan solid. ¹H NMR (DMSO-d₆) δ 13.44 (s, 1H) 11.10 (s, 1H) 8.52 (s, 1H) 8.04 (d, J=6.22 Hz, 1H) 7.66 (d, J=14.10 Hz, 1H) 7.59 (d, J=6.41 Hz, 1H) 7.41 7.24 (s, 1H) (s, 1H) 7.03 (d, J=17.03 Hz, 1H) 3.93 (m, 2H) 3.82 (m, 2H) 3.36 (m, 3H) 3.08 (m, 4H) 1.94 (s, 2H). MS m/z 315 (M+1).

### Example 26

### (2E)-N-[2-(4-chlorophenyl)ethyl]-3-(1H-indazol-3-yl)prop-2-enamide

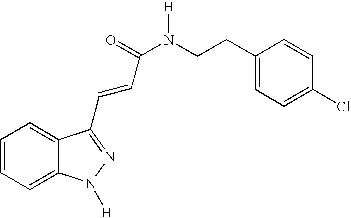

Utilizing the method described in Example 1 and Scheme 1, Method B, 2-(4-chlorophenyl)ethylamine (89.3 mg, 0.874 mmol) gave (2E)-N-[2-(4-chlorophenyl)ethyl]-3-(1H-indazol-3-yl)prop-2-enamide (16 mg) as a tan solid. ¹H NMR (DMSO-d₆) δ 13.41 (s, 1H) 8.22 (t, J=5.77 Hz, 1H) 8.00 (d, J=8.06 Hz, 1H) 7.63 (d, J=16.11 Hz, 1H) 7.58 (d, J=8.42 Hz, 1H) 7.35 (m, 2H) 7.41 (m, 1H) 7.28 (d, J=8.42 Hz, 2H) 7.24 (m, 1H) 6.97 (d, J=16.11 Hz, 1H) 3.44 (m, 2H) 2.79 (t, J=7.05 Hz, 2H). MS m/z 326 (M+1).

### Example 27

### Ethyl 1-[(2E)-3-(1H-indazol-3-yl)prop-2-enoyl]piperidine-4-carboxylate

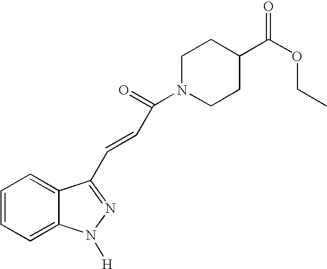

A mixture of (2E)-3-(1H-indazol-3-yl)prop-2-enoic acid (See Scheme 1, Method A—0.075 g, 0.40 mmol), PS-Carbodiimide (0.83 g, 0.80 mmol), HOBT (0.081 g, 0.60 mmol), and ethyl piperidine-4-carboxylate (0.052 g, 0.33 mmol) in 5 mL of DMF was stirred at room temperature for 18 hours. MP-Carbonate (0.687 g, 2.19 mmol) was added and the mixture was stirred for an additional 3 hours. The mixture was filtered and the DMF was removed in vacuo. Diethyl ether (15 mL) was added and the mixture was stirred at room temperature for 48 hours. The solid was collected by vacuum filtration and air dried to give ethyl 1-[(2E)-3-(1H-indazol-3-yl)prop-2-enoyl]piperidine-4-carboxylate (0.060 g) as a white solid. ¹H NMR (DMSO-d₆) δ 13.47 (s, 1H) 8.14 (d, J=8.20 Hz, 1H) 7.75 (d, J=15.53 Hz, 1H) 7.58 (d, J=8.35 Hz, 1H) 7.41 (m, 1H) 7.33 (d, J=15.67 Hz, 1H) 7.22 (m, 1H) 4.26 (d, J=44.39 Hz, 1H) 4.07 (q, J=7.18 Hz, 2H) 3.31 (m, 2H) 2.88 (m, 1H) 2.64 (m, 1H) 1.90 (m, 2H) 1.52 (m, 2H) 1.18 (t, J=7.10 Hz, 3H). MS m/z 328 (M+1).

### Example 28

### 3-[(1E)-3-(4-benzylpiperidin-1-yl)-3-oxoprop-1-enyl]-1H-indazole

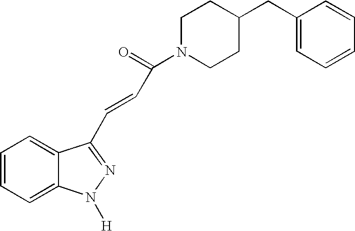

Utilizing the procedure of Example 27 and Scheme 1, Method A, 4-benzylpiperidine (58 mg, 0.33 mmol) gave 3-[(1E)-3-(4-benzylpiperidin-1-yl)-3-oxoprop-1-enyl]-1H-indazole (62 mg) as a white solid. ¹H NMR (DMSO-d₆) δ 13.42 (s, 1H) 8.11 (d, J=8.20 Hz, 1H) 7.74 (d, J=15.67 Hz, 1H) 7.57 (d, J=8.35 Hz, 1H) 7.40 (m, 1H) 7.32 (d, J=15.38 Hz, 1H) 7.21 (m, 6H) 4.48 (d, J=12.01 Hz, 1H) 4.22 (d, J=12.16 Hz, 1H) 3.05 (m, 1H) 2.62 (m, 1H) 2.53 (d, J=7.18 Hz, 2H) 1.80 (s, 1H) 1.64 (d, J=12.45 Hz, 2H) 1.11 (m, 2H). MS m/z 346 (M+1).

### Example 29

### (2E)-N-ethyl-3-(1H-indazol-3-yl)-N-(pyridin-4-yl-methyl)prop-2-enamide

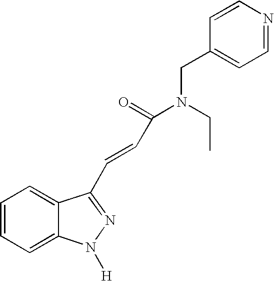

Utilizing the procedure of Example 27 and Scheme 1, Method A, N-ethyl-N-(pyridin-4-ylmethyl)amine (45 mg, 0.33 mmol) gave (2E)-N-ethyl-3-(1H-indazol-3-yl)-N-(pyridin-4-ylmethyl)prop-2-enamide (48 mg) as a tan solid. ¹H NMR (DMSO-d₆) δ 13.45 (m, 1H) 8.53 (m, 2H) 7.86 (m, 2H) 7.57 (m, 1H) 7.40 (m, 2H) 7.27 (m, 2H) 7.10 (m, 1H) 4.78 (m, 2H) 3.56 (m, 2H) 1.15 (m, 3H). MS m/z 307 (M+1).

### Example 30

### 8-[(2E)-3-(1H-indazol-3-yl)prop-2-enoyl]-1-phenyl-1,3,8-triazaspiro[4.5]decan-4-one

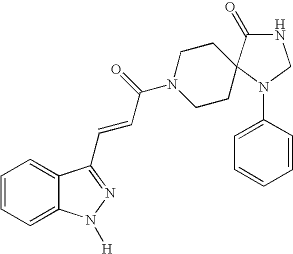

Utilizing the procedure of Example 27 and Scheme 1, Method A, 1-phenyl-1,3,8-triazaspiro[4.5]decan-4-one (76 mg, 0.33 mmol) gave 8-[(2E)-3-(1H-indazol-3-yl)prop-2-enoyl]-1-phenyl-1,3,8-triazaspiro[4.5]decan-4-one (81 mg) as a white solid. ¹H NMR (DMSO-d₆) δ 13.46 (s, 1H) 8.82 (s, 1H) 8.16 (d, J=8.35 Hz, 1H) 7.83 (d, J=15.67 Hz, 1H) 7.58 (d, J=8.50 Hz, 1H) 7.42 (m, 2H) 7.21 (m, 3H) 6.73 (m, 3H) 4.62 (s, 2H) 4.46 (d, J=11.28 Hz, 1H) 4.26 (d, J=10.25 Hz, 1H) 3.85 (m, 1H) 3.43 (m, 1H) 2.44 (s, 2H) 1.76 (s, 2H). MS m/z 402 (M+1).

### Example 31

### 3-[(1E)-3-oxo-3-(4-pyrazin-2-ylpiperazin-1-yl)prop-1-enyl]-1H-indazole

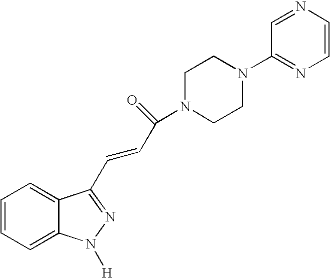

Utilizing the procedure described in Example 27 and Scheme 1, Method A, 2-piperazin-1-ylpyrazine (54 mg, 0.33 mmol) gave 3-[(1E-3-oxo-3-(4-pyrazin-2-ylpiperazin-1-yl)prop-1-enyl]-1H-indazole (74 mg) as a white solid. ¹H NMR (DMSO-d₆) δ 13.48 (s, 1H) 8.35 (d, J=1.32 Hz, 1H) 8.18 (d, J=8.06 Hz, 1H) 8.11 (dd, J=2.64, 1.46 Hz, 1H) 7.86 (d, J=2.64 Hz, 1H) 7.81 (d, J=15.67 Hz, 1H) 7.59 (d, J=8.35 Hz, 1H) 7.42 (m, 2H) 7.25 (m, 1H) 3.86 (s, 2H) 3.70 (m, 6H). MS m/z 335 (M+1).

### Example 32

### Methyl 4-(((2E)-3-(1H-indazol-3-yl)prop-2-enoyl)amino)benzoate

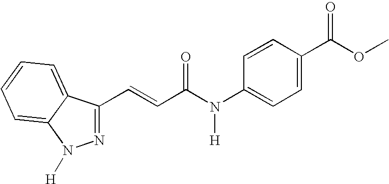

A solution of acryloyl chloride (2.38 g, 26.3 mmol) in CH₂Cl₂ (50 mL) was treated with methyl 4-amino benzoate (3.98 g, 26.3 mmol) and triethylamine (3.67 mL, 26.3 mmol) and stirred at ambient temperature for 1 hour. The solvent was evaporated and the residue was dissolved in DMF (60 mL). To this solution was added N-Boc-3-iodoindazole (5.16 g, 15 mmol), triethylamine (6.3 mL, 45 mmol), Pd(OAc)₂ (101 mg, 0.45 mmol), and tri-o-tolylphosphine (411 mg, 1.35 mmol) and the mixture was heated at 100° C. for 18 hours. The reaction mixture was cooled and partitioned between EtOAc (200 mL) and H₂O (200 mL). The organic layer was washed with 0.1 N HCl (150 mL) and brine (150 mL), dried (MgSO₄), filtered, and concentrated to give a brown oil. Purification by chromatography on silica gel eluting with 50:50 EtOAc:Hexane gave methyl 4-(((2E)-3-(1H-indazol-3-yl)prop-2-enoyl)amino)benzoate as tan solids (2.33 g). ¹H NMR (DMSO-d₆) δ 13.58 (s, 1H), 10.58 (s, 1H), 8.09 (d, 1H, J=7.9 Hz), 7.90 (m, 5H), 7.62 (d, 1H, J=8.4 Hz), 7.45 (t, 1H, J=7.1 Hz), 7.3 (m, 2H), 3.83 (s, 3H). MS m/z 322 (M+1).

### Example 33

### 4-(((2E)-3-(1H-indazol-3-yl)prop-2-enoyl)amino)benzoic acid

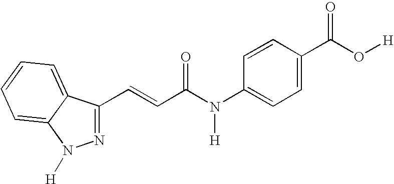

A solution of methyl 4-(((2E)-3-(1H-indazol-3-yl)prop-2-enoyl)amino)benzoate (Example 32-2.1 g, 6.5 mmol) in dioxane (20 mL) was treated with 1 N aqueous lithium hydroxide (19.5 mL, 19.5 mmol) and stirred at ambient temperature for 18 hours. The reaction mixture was acidified with 2N HCl. The precipitate obtained was collected by filtration, washed with Et₂O, and dried to give 4-(((2E)-3-(1H-indazol-3-yl)prop-2-enoyl)amino)benzoic acid as an off-white solid (1.98 g). ¹H NMR (DMSO-d₆) δ 13.50 (broad NH), 10.61 (s, 1H), 8.10 (d, 1H, J=8.1 Hz), 7.85 (m, 5H), 7.62 (m, 1H), 7.45 (t, 1H, J=7 Hz), 7.28 (m, 2H), 4.00 (broad OH).

### Example 34

### Methyl 3-(((2E)-3-(1H-indazol-3-yl)prop-2-enoyl)amino)benzoate

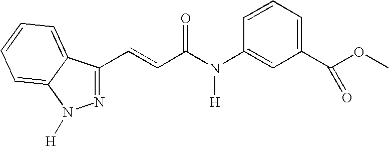

Utilizing the procedure described in Example 32 and Scheme 1, Method B methyl 3-amino benzoate (3.98 g, 26.3 mmol) gave methyl 3-(((2E)-3-(1H-indazol-3-yl)prop-2-enoyl)amino)benzoate. ¹H NMR (DMSO-d₆) δ 13.56 (s, 1H), 10.48 (s, 1H), 8.40 (s, 1H), 8.08 (d, 1H, J=8 Hz), 7.97 (d, 1H, J=8.9 Hz), 7.85 (d, 1H, J=15.9 Hz), 7.61 (m, 2H), 7.51 (m, 2H), 7.31 (t, 1H, J=7.7 Hz), 7.20 (d, 1H, J=16 Hz), 3.88 (s, 3H). MS m/z 322 (M+1).

### Example 35

### 3-(((2E)-3-(1H-indazol-3-yl)prop-2-enoyl)amino)benzoic acid

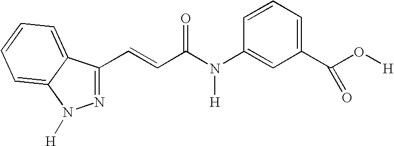

Utilizing the procedure described in Example 33, Methyl 3-(((2E)-3-(1H-indazol-3-yl)prop-2-enoyl)amino)benzoate (prepared in Example 34-2.1 g, 6.5 mmol) gave 3-(((2E)-3-(1H-indazol-3-yl)prop-2-enoyl)amino)benzoic acid as an off-white solid (1.88 g). H NMR (DMSO-d₆) δ 13.5 (broad NH), 10.50 (s, 1H), 8.35 (s, 1H), 8.10 (d, 1H, J=8.2 Hz), 7.98 (d, 1H, J=8.1 Hz), 7.83 (d, 1H, J=15.8 Hz), 7.64 (t, 2H, J=7.9 Hz), 7.48 (m, 2H), 7.26 (m, 2H), 4.22 (broad OH).

### Example 36

### 4-(((2E)-3-(1H-indazol-3-yl)prop-2-enoyl)amino)-N-(2-pyridin-3-ylethyl)benzamide

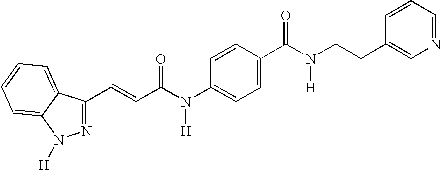

A solution of 4-(((2E)-3-(1H-indazol-3-yl)prop-2-enoyl)amino)benzoic acid (Example 33-75 mg, 0.24 mmol), HOBT (49 mg, 0.36 mmol) and polymer-supported carbodiimide (Argonaut Technologies, catalogue #800371, 500 mg, 0.96 mmol/g, 0.48 mmol) in DMF (2 mL) was stirred at ambient temperature for 15 minutes, and then 2-pyridin-3-ylethylamine (24 mg, 0.2 mmol) was added. The reaction mixture was shaken for 18 hours, and then MP-carbonate (Argonaut Technologies, catalogue #800269, 3.19 mmol/gram, 1.1 mmol, 345 mg) was added, and the reaction shaken an additional 8 hours. The reaction mixture was filtered to remove the resins, and the resins washed with EtOAc (2 mL). The combined filtrate was concentrated to dryness, the residue was treated with Et₂O (3 mL), and the suspension stirred for 18 h. The suspension was filtered and the resulting solids dried to afford 4-(((2E)-3-(1H-indazol-3-yl)prop-2-enoyl)amino)-N-(2-pyridin-3-ylethyl)benzamide as an off-white powder (16 mg). ¹H NMR (DMSO-d₆) δ 13.56 (s, 1H), 10.46 (s, 1H), 8.46 (m, 3H), 8.09 (d, 1H, J=8.7 Hz), 7.81 (m, 4H), 7.64 (m, 2H), 7.46 (t, 1H, J=8 Hz), 7.30 (m, 4H), 3.51 (q, 2H, J=6 Hz), 2.87 (t, 2H, J=7 Hz). MS m/z 412 (M+1).

### Example 37

### 4-(((2E)-3-(1H-indazol-3-yl)prop-2-enoyl)amino)-N-methyl-N-(2-pyridin-1-ylethyl)benzamide

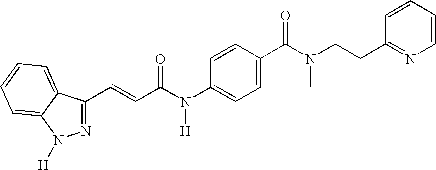

Utilizing the procedure of Example 36, reaction of 4-(((2E)-3-(1H-indazol-3-yl)prop-2-enoyl)amino)benzoic acid (75 mg, 0.24 mmol) and N-methyl-2-pyridin-2-ylethanamine (27 mg, 0.2 mmol) to yield 4-(((2E)-3-(1H-indazol-3-yl)prop-2-enoyl)amino)-N-methyl-N-(2-pyridin-2-ylethyl)benzamide as an off-white powder (18 mg). ¹H NMR (DMSO-d₆) δ 13.56 (s, 1H), 10.40 (s, 1H), 8.5 (m, 2H), 8.10 (d, 1H, J=8 Hz), 7.83 (d, 1H, J=16 Hz), 7.71 (m, 3H), 7.61 (d, 1H, J=8.3 Hz), 7.46 (t, 1H, J=7.1 Hz), 7.25 (m, 5H), 3.7 (m, 3H), 2.97 (m, 4H). MS m/z 426 (M+1).

### Example 38

### 4-(((2E)-3-(1H-indazol-3-yl)prop-2-enoyl)amino)-N-methyl-N-(1-methylpyrrolidin-3-yl)benzamide

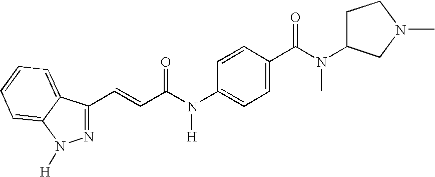

Utilizing the procedure of Example 36, reaction of 4-(((2E)-3-(1H-indazol-3-yl)prop-2-enoyl)amino)benzoic acid (75 mg, 0.24 mmol) and N,1-dimethylpyrrolidin-3-amine (23 mg, 0.2 mmol) yielded 4-(((2E)-3-(1H-indazol-3-yl)prop-2-enoyl)amino)-N-methyl-N-(1-methylpyrrolidin-3-yl)benzamide as an off-white powder (16 mg). ¹H NMR (DMSO-d₆) δ 13.55 (s, 1H), 10.41 (s, 1H), 8.08 (d, 1H, J=8.2 Hz), 7.8 (m, 3H), 7.62 (d, 1H, J=8.4 Hz), 7.32 (m, 5H), 3.36 (m, 2H), 2.9 (s, 3H), 2.72 (m, 2H), 2.53 (m, 2H), 2.23 (s, 3H), 1.82 (m, 1H). MS m/z 404 (M+1).

### Example 39

### 3-(((2E)-3-(1H-indazol-3-yl)prop-2-enoyl)amino)-N-(2-morpholin-4-ylethyl)benzamide

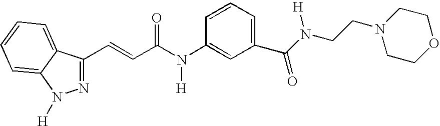

Utilizing the procedure of Example 36, reaction of 3-(((2E)-3-(1H-indazol-3-yl)prop-2-enoyl)amino)benzoic acid (75 mg, 0.24 mmol) and 2-morpholin-4-ylethanamine (26 μL, 0.2 mmol) to yield 3-(((2E)-3-(1H-indazol-3-yl)prop-2-enoyl)amino)-N-(2-morpholin-4-ylethyl)benzamide as an off-white powder (18 mg). ¹H NMR (DMSO-d₆) δ 13.54 (s, 1H), 10.40 (s, 1H), 8.39 (t, 1H, J=5.6 Hz), 8.11 (m, 2H), 7.9 (d, 1H, J=7.9 Hz), 7.81 (d, 1H, J=15.9 Hz), 7.61 (d, 1H, J=8.4 Hz), 7.45 (m, 3H), 7.24 (m, 2H), 3.57 (m, 4H), 3.37 (m, 3H), 2.41 (m, 5H). MS m/z 420 (M+1).

### Example 40

### (2E)-N-(3-((4-benzylpiperazin-1-yl)carbonyl)phenyl)-3-(1H-indazol-3-yl)prop-2-enamide

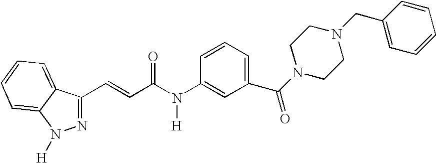

Utilizing the procedure of Example 36, reaction of 3-(((2E)-3-(1H-indazol-3-yl)prop-2-enoyl)amino)benzoic acid (75 mg, 0.24 mmol) and 1-benzyl piperazine (35 μL, 0.2 mmol) to yield (2E)-N-(3-((4-benzylpiperazin-1-yl)carbonyl)phenyl)-3-(1H-indazol-3-yl)prop-2-enamide as a tan powder (13 mg). ¹H NMR (DMSO-d₆) δ 13.54 (s, 1H), 10.37 (s, 1H), 8.07 (d, 1H, J=8.2 Hz), 7.81 (m, 2H), 7.65 (m, 2H), 7.4 (m, 2H), 7.3 (m, 7H), 3.5 (s, 2H), 3.38 (m, 4H), 2.39 (m, 4H). MS m/z 466 (M+1).

### Example 41

### N-ethyl-3-(((2E)-3-(1H-indazol-3-yl)prop-2-enoyl)amino)-N-(pyridin-4-ylmethyl)benzamide

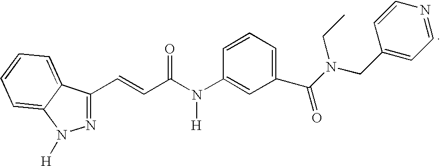

Utilizing the procedure described in Example 36, reaction of 3-(((2E)-3-(1H-indazol-3-yl)prop-2-enoyl)amino)benzoic acid (75 mg, 0.24 mmol) and N-ethyl-N-(pyridin-4-ylmethyl)amine (28 μL, 0.2 mmol) to yield N-ethyl-3-(((2E)-3-(1H-indazol-3-yl)prop-2-enoyl)amino)-N-(pyridin-4-ylmethyl)benzamide as an off-white powder (12 mg). ¹H NMR (DMSO-d₆) δ 13.56 (s, 1H), 12.97 (br s, 1H), 8.56 (m, 2H), 8.09 (d, 1H, J=8.1 Hz), 7.70 (m, 4H), 7.46 (m, 2H), 7.25 (m, 5H), 4.6 (br s, 2H), 3.39 (q, 2H, J=7 Hz), 1.09 (t, 3H, J=6.9 Hz). MS m/z 426 (M+1).

### BIOLOGICAL DATA

### SGK-1 Enzyme Assay (SGK1e)

Compounds of the present invention were tested for Serum Glucocorticoid-regulated Kinase-1 (SGK-1) protein serine/threonine kinase inhibitory activity in substrate phosphorylation assays using enzyme purified from a baculovirus expression vector system. The recombinant baculovirus was made to express the intracellular domain of SGK-1 (GenBank accession number MD41091). The virus expressed a truncated form of the human enzyme that included amino acids 61-431. Serine422 was replaced with Aspartic acid to activate the enzyme, and 6 Histidine residues were added at the amino terminus to facilitate purification. The protein was purified using Ni-NTA agarose affinity chromatography. The peptide substrate was an N-terminal biotinylated sunthetic peptide named Crosstide (biotin-Ahx-GRPRTSSFAEG-OH), corresponding to the sequence in GSK3 surrounding the serine phosphorylated by MAPKAP Kinase-1/Rsk and p70 S6 Kinase.

The method measures the ability of the isolated enzyme to catalyze the transfer of the γ-phosphate from ATP onto serine/threonine residues in the biotinylated Crosstide. Reactions were performed in 96-well polystyrene round-bottom plates in a final volume of 30 uL. Reaction mixtures contained 62.5 mM HEPES (pH 7.4), 10 mM MgCl₂, 0.1 mM EDTA, 0.0024% TWEEN-20, and 1 mM DTT (added fresh daily), 10 μM ATP, 0.2 μCi [[v-³³P] ATP per reaction, 4 μM Crosstide peptide substrate, and 1 nM SGK1 enzyme. Reactions were initiated by adding the indicated enzyme. The reaction was allowed to proceed for 2 hours, then terminated by the addition of 50 mM EDTA and quantified using a scintillation proximity assay procedure as described (McDonald, O. B., Antonsson, B., Arkinstal, S., Marshall, C. J., and Wood, E. R. (1999) Analytical Biochemistry, 268, 318-329).

Compounds under analysis were dissolved in Me₂SO to 1 mM and serially diluted 1 to 3 with Me₂SO through eleven columns of a 96 well plate. 1 uL of each concentration was transferred to the corresponding well of the assay plate. This created a final compound concentration range from 0.56 nM to 33.3 uM.

The data for dose responses were plotted as % Control calculated with the data reduction formula 100*(U1-C2)/(C1-C2) versus concentration of compound and fitted to the curve described by:

γ=((Vmax*x)/(K+x))

where Vmax is the upper asymptote and K is the IC₅₀

All exemplified Examples 1-41 were run with the recited assay and showed inhibitory activity versus SGK-1 with a pIC₅₀ of 4.0 or greater. Representative Examples are depicted in Table I.

TABLE I

| Ex. No | SGK1e |
|---|---|
| 1 | +++ |
| 3 | +++ |
| 12 | +++ |
| 15 | +++ |
| 19 | +++ |
| 24 | ++ |
| 27 | ++ |
| 30 | +++ |
| 36 | +++ |
| 41 | +++ |

+ = pIC₅₀ of 4.0-5.0;
++ = pIC₅₀ of 5.0-6.0;
+++ = pIC₅₀ of >6.0;

What is claimed is:

1. A compound of Formula (I): 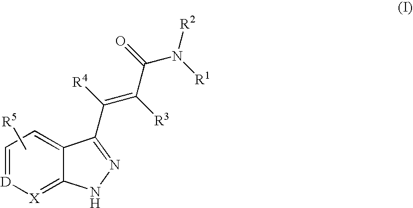
or a salt, solvate, or physiologically functional derivative thereof:
wherein:
D is C—R and X is N, or
D is N and X is C—R, or
D is C—R and X is C—R,
where each R is independently selected from hydrogen, halo, cyano, or C₁-C₆ alkyl;
R¹ is a group defined by -(Q)ₘ-(Q¹)ₙ-(Q²)ₚ, wherein;
Q is arylene or heteroarylene, and m is 0 or 1,
Q¹ is O(CH₂)_{q}, (CH₂)ᵣC(O), or S(O)₂, and
n is 0 or 1,
q is 0, 1, 2, 3, or 4, and
r is 1, 2, 3, or 4,
Q² is C₁-C₆ alkyl, C₃-C₇ cycloalkyl, —OH, C₁-C₃ alkoxy, NR⁶R⁷, aryl, aryloxy, heteroaryl, heterocyclyl, or R⁹R¹⁰, and p is 0 or 1
R² is —H, or C₁-C₆alkyl; or
R¹ and R² together with the nitrogen to which they are attached form a ring system, said ring system being a substituted or unsubstituted heterocyclyl or heterocyclic spiro ring system;
R³ is —H or C₁-C₃ alkyl;
R⁴ is —H or C₁-C₃ alkyl;
R⁵ is —H, halo, —CN, —OH, C₁-C₃ alkyl, C₁-C₃alkoxy, —NO₂, aryl, or NR′R″;
R⁶ is —H or C₁-C₆ alkyl;
R⁷ is —H, C₁-C₆ alkyl, C₃-C₇ cycloalkyl, —R⁹R¹⁰, aralkyl, heterocyclyl, or —C(O)R⁸;
R⁸ is hydrogen or C₁-C₆ alkyl;
R⁹ is C₁-C₆ alkylene or heterocyclylene;
R¹⁰ is C₁-C₆ alkoxy, aryl, aralkyl, heteroaryl, aryloxy, heterocyclyl, —C(O)OR⁸, —C(O)R⁸, or —C(O)NR′R″;
R′ is —H or C₁-C₆ alkyl;
R″ is —H, C₁-C₆ alkyl, —C(O)R′″, —S(O)₂R′″, or C(O)NHR′″; and
R′″ is C₁-C₆ alkyl, aryl, aralkyl, heteroaryl, or heterocyclyl.

2. A compound as claimed in claim 1, wherein D is N.

3. A compound as claimed in claim 1, wherein D is C—R and R is H.

4. A compound as claimed in claim 1, wherein X is N.

5. A compound as claimed in claim 1, wherein X is C—R and R is H.

6. A compound as claimed in claim 1, wherein D is N, X is C—R, and R is H.

7. A compound as claimed in claim 1, wherein D is C—R, R is H, and X is N.

8. A compound as claimed in claim 1, wherein X is C—R, D is C—R, and each R is H.

9. A compound as claimed in claim 1, wherein m and n are 0, p is 1 and R¹ is -(Q²).

10. A compound as claimed in claim 1, wherein m and p are 1, n is O and R¹ is -(Q)-(Q²).

11. A compound as claimed in claim 1, wherein m, n, and p are 1, and R¹ is -(Q)-(Q¹)-(Q²).

12. A compound as claimed in claim 1, wherein m and p are 1 and Q is arylene.

13. A compound as claimed in claim 1, wherein m and p are 1 and Q is heteroarylene.

14. A compound as claimed in claim 1, wherein n and p are 1 and Q¹ is O(CH₂)_{q}, where q is 0, 1, 2, 3, or 4.

15. A compound as claimed in claim 1, wherein n and p are 1 and Q¹ is (CH₂)ᵣ and where r is 1, 2, 3, or 4.

16. A compound as claimed in claim 1, wherein n and p are 1 and Q¹ is C(O).

17. A compound as claimed in claim 1, wherein n and p are 1 and Q¹ is S(O)₂.

18-39. (canceled)

40. A compound as claimed in claim 1, selected from the group consisting of:
(2E)-N-(1,3-benzothiazol-6-yl)-3-(1H-indazol-3-yl)-2-propenamide;
(2E)-N-(3,4-dimethyl-5-isoxazolyl)-3-(1H-indazol-3-yl)-2-propenamide;
(2E)-N-(2-cyanophenyl)-3-(1H-indazol-3-yl)-2-propenamide;
(2E)-3-(1H-indazol-3-yl)-N-(6-methoxy-3-pyridinyl)-2-propenamide;
(2E)-N-(3-chlorophenyl)-3-(1H-indazol-3-yl)-2-propenamide;
(2E)-N-(2,3-dihydro-1H-inden-5-yl)-3-(1H-indazol-3-yl)-2-propenamide;
(2E)-N-[4-(dimethylamino)phenyl]-3-(1H-indazol-3-yl)-2-propenamide;
(2E)-N-(3-cyclopropyl-1-methyl-1H-pyrazol-5-yl)-3-(1H-indazol-3-yl)-2-propenamide;
(2E)-3-(1H-indazol-3-yl)-N-(5-quinolinyl)-2-propenamide;
(2E)-N-[3-(acetylamino)phenyl]-3-(1H-indazol-3-yl)-2-propenamide;
(2E)-3-(1H-indazol-3-yl)-N-(3,4,5-trimethoxyphenyl)-2-propenamide;
(2E)-N-(3-benzoylphenyl)-3-(1H-indazol-3-yl)-2-propenamide;
(2E)-N-[3-chloro-4-(4-morpholinyl)phenyl]-3-(1H-indazol-3-yl)-2-propenamide;
(2E)-N-{5-[(diethylamino)sulfonyl]-2-methoxyphenyl}-3-(1H-indazol-3-yl)-2-propenamide;
(2E)-N-{4-[2-(diisopropylamino)ethoxy]-3-methoxyphenyl}-3-(1H-indazol-3-yl)-2-propenamide;
(2E)-3-(1H-indazol-3-yl)-N-(3-methoxy-4-{2-[(2-phenoxyethyl)amino]ethoxy}phenyl)-2-propenamide;
(2E)-3-(1H-indazol-3-yl)-N-{3-methoxy-4-[2-(4-morpholinyl)ethoxy]phenyl}-2-propenamide;
(2E)-3-(1H-indazol-3-yl)-N-(3-methoxy-4-{2-[(2-methoxyethyl)amino]-ethoxy}phenyl)-2-propenamide;
(2E)-3-(1H-indazol-3-yl)-N-(3-methoxy-4-{2-[methyl(propyl)amino]ethoxy}phenyl)-2-propenamide;
(2E)-N-[4-(2-{[2-(4-chlorophenyl)ethyl]amino]ethyoxy)-3-methoxyphenyl}-3-(1H-indazol-3-yl)-2-propenamide;
ethyl 4-{[2-(4-{[(2E)-3-(1H-indazol-3-yl)-2-propenoyl]amino}-2-methoxyphenoxy)ethyl]amino}-1-piperidine carboxylate;
(2E)-N-{4-[2-(4-acetyl-1-piperazinyl)ethoxy]-3-methoxyphenyl}-3-(1H-indazol-3-yl)-2-propenamide;
(2E)-N-benzyl-3-(1H-indazol-3-yl)prop-2-enamide;
(2E)-3-(1H-indazol-3-yl)-N-isobutylprop-2-enamide;
(2E)-3-(1H-indazol-3-yl)-N-(3-morpholin-4-ylpropyl)prop-2-enamide;
(2E)-N-[2-(4-chlorophenyl)ethyl]-3-(1H-indazol-3-yl)prop-2-enamide;
ethyl 1-[(2E)-3-(1H-indazol-3-yl)prop-2-enoyl]piperidine-4-carboxylate;
3-[(1E)-3-(4-benzylpiperidin-1-yl)-3-oxoprop-1-enyl]-1H-indazole;
(2E)-N-ethyl-3-(1H-indazol-3-yl)-N-(pyridin-4-ylmethyl)prop-2-enamide;
8-[(2E)-3-(1H-indazol-3-yl)prop-2-enoyl]-1-phenyl-1,3,8-triazaspiro[4.5]decan-4-one;
3-[(1E)-3-oxo-3-(4-pyrazin-2-ylpiperazin-1-yl)prop-1-enyl]-1H-indazole;
METHYL 4-(((2E)-3-(1H-INDAZOL-3-YL)PROP-2-ENOYL)AMINO)BENZOATE:
4-(((2E)-3-(1H-indazol-3-yl)prop-2-enoyl)amino)benzoic acid;
methyl 3-(((2E)-3-(1H-indazol-3-yl)prop-2-enoyl)amino)benzoate;
3-(((2E)-3-(1H-indazol-3-yl)prop-2-enoyl)amino)benzoic acid;
4-(((2E)-3-(1H-indazol-3-yl)prop-2-enoyl)amino)-N-(2-pyridin-3-ylethyl)benzamide;
4-(((2E)-3-(1H-indazol-3-yl)prop-2-enoyl)amino)-N-methyl-N-(2-pyridin-2-ylethyl)benzamide;
4-(((2E)-3-(1H-indazol-3-yl)prop-2-enoyl)amino)-N-methyl-N-(1-methylpyrrolidin-3-yl)benzamide;
3-(((2E)-3-(1H-indazol-3-yl)prop-2-enoyl)amino)-N-(2-morpholin-4-ylethyl)benzamide;
(2E)-N-(3-((4-benzylpiperazin-1-yl)carbonyl)phenyl)-3-(1H-indazol-3-yl)prop-2-enamide; and
N-ethyl-3-(((2E)-3-(1H-indazol-3-yl)prop-2-enoyl)amino)-N-(pyridin-4-ylmethyl)benzamide
or a salt, solvate, or physiologically functional derivative thereof.

41. A pharmaceutical composition, comprising: a therapeutically effective amount of a compound as claimed in claim 1, or a salt, solvate, or a physiologically functional derivative thereof and one or more of pharmaceutically acceptable carriers, diluents and excipients.

42. A method of treating a disorder in a mammal, said disorder being mediated by at least one of inappropriate SGK-1 activity, comprising: administering to said mammal a therapeutically effective amount of a compound as claimed in claim 1, or a salt, solvate, or a physiologically functional derivative thereof.

43-44. (canceled)
